(19) United States
(12) Patent Application Publication
(10) Pub. No.: US 2026/0183500 A1
(43) Pub. Date: Jul. 2, 2026

(54) A BREATHING ASSISTANCE APPARATUS FOR PROVIDING RESPIRATORY THERAPY

(71) Applicant: Fisher & Paykel Healthcare Limited, Auckland (NZ)

(72) Inventors: Malik Tivanka Rajiv PEIRIS, Auckland (NZ); Simei Gomes WYSOSKI, Auckland (NZ)

(21) Appl. No.: 19/108,565

(22) PCT Filed: Sep. 14, 2023
(86) PCT No.: PCT/IB2023/059109

§ 371 Date: Mar. 4, 2025

Related U.S. Application Data
(60) Provisional application No. 63/375,677, filed on Sep. 14, 2022.

(51) Int. Cl. A61M 16/00; A61M 16/06; A61M 16/10

(52) U.S. Cl. CPC A61M 16/0051; A61M 16/0096; A61M 16/0666; A61M 16/1095; A61M 2016/0027; A61M 2016/0033; A61M 2205/15; A61M 2205/3368; A61M 2205/3375; A61M 2205/581; A61M 2205/583; A61M 2230/42; A61M 2230/43; A61M 2240/00

(57) ABSTRACT

Disclosed is a breathing assistance apparatus configured to deliver a respiratory therapy to a patient, the breathing assistance apparatus having a flow generator, a humidifier, and a controller configured to control the breathing assistance apparatus. The breathing assistance apparatus is changeable between a plurality of therapy modes.

### FIELD OF THE DISCLOSURE

The present disclosure relates to methods and systems for providing a respiratory therapy to a patient. In particular, the present disclosure relates to using a breathing assistance apparatus to provide various types of respiratory therapy.

### BACKGROUND

Breathing assistance apparatuses are used in various environments such as hospital, medical facility, residential care, or home environments to deliver a flow of gases to users or patients. A breathing assistance or respiratory therapy apparatus (collectively, “respiratory apparatus” or “respiratory devices”) may be used to deliver supplementary oxygen or other gases with a flow of gases, and/or a humidification apparatus to deliver heated and humidified gases. A breathing assistance apparatus may allow adjustment and control over characteristics of the gases flow, including flow rate, temperature, gases concentration, humidity, pressure, etc. Sensors, such as flow sensors and/or pressure sensors, are used to measure characteristics of the gases flow.

### SUMMARY

In some locations, a patient may need to be provided with multiple therapy types. The present disclosure provides systems and methods of providing a single apparatus capable of providing multiple therapy types. The flow generator can also include an integrated humidifier to heat and humidify the flow of gas. A heated breathing tube can also be used to deliver the flow of gas from the humidifier to the patient interface. The flow generator can also include an integrated blender to provide supplementary gases to the gases flow. The flow generator may be a flow generator that draws in ambient gases e.g. ambient air rather than be connected to a gases source e.g. a gas tank or a wall source. The blender allows a supplementary gas or gases to be mixed with the drawn in ambient gases.

The term breathing assistance apparatus and breathing assistance device can be interchangeably used to described and define the same item.

The breathing assistance apparatus may be part of a breathing assistance system comprising one or more additional components as described in more detail below (for example an inspiratory tube, an expiratory tube, a bubbler)

In an aspect there is disclosed a breathing assistance apparatus configured to deliver respiratory therapy to a patient, the breathing assistance apparatus comprising:
a flow generator, and a humidifier in fluid communication with the flow generator,
a controller configured to control the breathing assistance apparatus,
wherein the breathing assistance apparatus is changeable between a plurality of therapy modes, wherein the plurality of therapy modes comprise: a high flow therapy mode, a Bubble CPAP therapy mode, a variable flow CPAP mode, an asynchronous Nasal Intermittent Positive Pressure Ventilation mode and a synchronous Nasal Intermittent Positive Pressure Ventilation mode,
wherein:
   in the high flow therapy mode the breathing assistance apparatus is configured to provide high flow therapy,
   in the Bubble CPAP therapy mode the breathing assistance apparatus is configured to provide bubble CPAP therapy,
   in the variable flow CPAP mode the breathing assistance apparatus is configured to provide variable flow CPAP therapy,
   in the asynchronous Nasal Intermittent Positive Pressure Ventilation mode the breathing assistance apparatus is configured to provide asynchronous Nasal Intermittent Positive Pressure Ventilation therapy, and
   in the synchronous Nasal Intermittent Positive Pressure Ventilation mode the breathing assistance apparatus is configured to provide synchronous Nasal Intermittent Positive Pressure Ventilation therapy.

In some examples, the plurality of therapy modes comprises a Neurally Adjusted Ventilatory Assist (NIV-NAVA) mode.

In some examples, the plurality of therapy modes comprises a High Frequency Oscillatory Ventilation (HFOV) mode.

In some examples, the plurality of therapy modes comprises a volume-limited pressure control mode.

In some examples, the plurality of therapy modes comprises a volume control ventilation mode.

In some examples, the plurality of therapy modes comprises a resuscitation mode.

In some examples, the plurality of therapy modes comprises a High Frequency Percussive Ventilation (HFPV) mode.

In some examples, in the NIV-NAVA mode the breathing assistance apparatus is configured to provide NIV-NAVA therapy.

In some examples, in the HFOV mode the breathing assistance apparatus is configured to provide HFOV therapy.

In some examples, in the volume-limited pressure control mode the breathing assistance apparatus is configured to provide volume-limited pressure control therapy.

In some examples, in the volume control ventilation mode, the breathing assistance apparatus is configured to provide volume control ventilation therapy.

In some examples, in the resuscitation mode the breathing assistance apparatus is configured to provide resuscitation therapy.

In some examples, in the HFPV mode, the breathing assistance apparatus is configured to provide HFPV therapy.

In some examples, the apparatus is configured to be connected to and/or comprises at least one gases property sensor, the at least one gases property sensor, wherein the controller is configured to calculate a determined flow rate and/or a determined pressure of the gases in a gases flow pathway, based on at least an output of the at least one gases property sensor.

In some examples, the controller is configured to generate one or more alarms based on a or the determined flow rate and/or determined pressure.

In some examples, the one or more alarms are associated with the plurality of therapy modes.

In another aspect there is disclosed a breathing assistance apparatus configured to deliver respiratory therapy to a, the breathing assistance apparatus comprising:
a flow generator, and a humidifier in fluid communication with the flow generator,
a controller configured to control the breathing assistance apparatus,
   wherein the controller is configured to be in communication with at least one gases property sensor, wherein the controller is configured to calculate a determined flow rate and/or a determined pressure of the gases in a gases flow pathway based on an least an output of the at least one gases property sensor,
   wherein the controller is configured to generate one or more alarms based on the determined flow rate and/or determined pressure, wherein the one or more alarms comprise:
   a) An over pressure alarm,
   b) An elevated pressure alarm,
   c) A low pressure alarm,
   d) An excessive leak alarm,
   e) A blockage alarm,
   f) An apnea alarm,
   g) Any combination of a)-f).

In some examples, the one or more alarms comprise an intermittent or no bubbling alarm.

In some examples, the one or more alarms comprise a condensation alarm.

In some examples, the one or more alarms comprise an incorrect hardware alarm.

In some examples, the one or more alarms comprise a prong dislodge alarm.

In some examples, the one or more alarms comprise a loss of physiological signal alarm.

In some examples, the one or more alarms comprise a high/low breath rate alarm.

In some examples, the one or more alarms comprise a tidal volume alarm.

In some examples, the one or more alarms comprise an SpO2 alarm.

In some examples, the one or more alarms comprise an exhaled CO2 alarm.

In another aspect there is disclosed a breathing assistance apparatus configured to deliver respiratory therapy to a, the breathing assistance apparatus comprising:
a flow generator, and a humidifier in fluid communication with the flow generator,
a controller configured to control the breathing assistance apparatus,
   wherein the controller is configured to be in communication with at least one gases property sensor, wherein the controller is configured to calculate a determined flow rate and/or a determined pressure of the gases in a gases flow pathway based on an least an output of the at least one gases property sensor,
   wherein the controller is configured to generate one or more alarms based on the determined flow rate and/or determined pressure, wherein the one or more alarms comprise:
   a) An over pressure alarm,
   b) An elevated pressure alarm,
   c) A low pressure alarm,
   d) An excessive leak alarm,
   e) A blockage alarm,
   f) An apnea alarm,
   g) An intermittent or no bubbling alarm,
   h) A condensation alarm,
   i) An incorrect hardware alarm,
   j) A prong dislodge alarm,
   k) A loss of physiological signal alarm,
   l) A high/low breath rate alarm,
   m) A tidal volume alarm,
   n) An SpO2 alarm,
   o) An exhaled CO2 alarm,
   p) Any combination of a)-o).

In some examples, the apparatus is changeable between a plurality of therapy modes, wherein one or more alarms is associated with one or more of the plurality of therapy modes.

In some examples, the plurality of therapy modes comprise: a high flow therapy mode, a Bubble CPAP therapy mode, a variable flow CPAP mode, an asynchronous Nasal Intermittent Positive Pressure Ventilation mode and a synchronous Nasal Intermittent Positive Pressure Ventilation mode.

In some examples, the plurality of therapy modes comprise: a high flow therapy mode, a Bubble CPAP therapy mode, a variable flow CPAP mode, an asynchronous Nasal Intermittent Positive Pressure Ventilation mode and a synchronous Nasal Intermittent Positive Pressure Ventilation mode, a Non-Invasive Ventilation Neurally Adjusted Ventilatory Assist (NIV-NAVA) mode, a High Frequency Oscillatory Ventilation (HFOV) mode, a volume-limited pressure control mode, a volume control ventilation mode, and a resuscitation mode.

In some examples, the humidifier is in, or forms part of a gases flow path.

In some examples, an inspiratory conduit and/or expiratory conduit comprise a heater.

In some examples, the heater is a heater wire.

In some examples, the heater wire is located:
in a passageway of the inspiratory conduit and/or expiratory conduit,
attached to a wall of the inspiratory conduit and/or expiratory conduit,
embedded in a wall of the inspiratory conduit and/or expiratory conduit.

In some examples, when the one or more alarms is generated, the controller is configured to:
a) Disable the flow generator,
b) Disable a heater of the humidifier,
c) Disable a heater of an attached conduit,
d) Display a message on a display,
e) Generate an audio warning,
f) Generate a visual warning,
g) Any combination of a)-f).

In some examples, each of the plurality of therapy modes comprises an associated control program.

In some examples, a user can select a desired therapy mode from the plurality of therapy modes.

In some examples, the controller is configured to select and apply a program that corresponds to the selected mode.

In some examples, the control programs define:
a) therapy parameters
b) which of the one or more alarms applies to the therapy mode associated with the control program,
c) alarm conditions associated the one or more alarms
d) any associated alarm thresholds associated the one or more alarms.
e) Any combination of a)-e)

In some examples, the therapy parameters comprise:
a) A humidity level,
b) A set pressure or set pressures,
c) A set flow rate,
d) A gases temperature,
e) Any combination of a)-d).

In some examples, in the high flow therapy mode the apparatus, is configured to provide gases to the patient at a set flow rate.

In some examples, the high flow therapy is nasal high flow therapy.

In some examples, humidity is provided at a or the humidity level during high flow therapy.

In some examples, in the high flow therapy mode an unsealed patient interface coupled to a or the inspiratory conduit.

In some examples, the unsealed patient interface is a nasal cannula.

In some examples, the nasal cannula is positioned on a user's face to provide gases to the nares of the user.

In some examples, in the bubble CPAP mode, the apparatus is configured to provide gases to the patient at a set flow rate.

In some examples, in the bubble CPAP therapy mode, a sealed patient interface is coupled to a or the inspiratory conduit, and the inspiratory conduit is configured to be coupled to the apparatus, and an expiratory conduit coupled to the sealed patient interface.

In some examples, the expiratory conduit is coupled to a pressure regulator to regulate pressure within the patient interface and/or a patient's airways.

In some examples, the pressure regulator comprises a chamber with a column of water, wherein the expiratory conduit is submerged into the column of water.

In some examples, in the variable flow CPAP mode, the apparatus is configured to provide gases to the patient at a set pressure.

In some examples, in the variable flow CPAP mode, a sealed patient interface is coupled to a or the inspiratory conduit, and the inspiratory conduit is configured to be coupled to the apparatus, and an expiratory conduit coupled to the sealed patient interface.

In some examples, the expiratory conduit is coupled to a pressure regulator to regulate pressure within the patient interface and/or the patient's airways.

In some examples, the pressure regulator comprises an expiratory bias orifice.

In some examples, in the asynchronous Nasal Intermittent Positive Pressure Ventilation mode and/or the synchronous Nasal Intermittent Positive Pressure Ventilation mode, a sealed patient interface is coupled to the inspiratory conduit, and the inspiratory conduit is configured to be coupled to the apparatus.

In some examples, in the asynchronous Nasal Intermittent Positive Pressure Ventilation mode and/or the synchronous Nasal Intermittent Positive Pressure Ventilation mode an expiratory conduit is coupled to the sealed patient interface.

In some examples, in the asynchronous Nasal Intermittent Positive Pressure Ventilation mode, the apparatus is configured to provide gases to the patient as a breathing cycle comprising a set inspiratory airway pressure to cause inspiration of the patient, and a set expiratory airway pressure to cause expiration of the patient.

In some examples, the breathing cycle has a set time period.

In some examples, in the asynchronous Nasal Intermittent Positive Pressure Ventilation mode, the apparatus is configured to provide gases to the patient at the set inspiratory airway pressure and the set expiratory airway pressure irrespective of detection of a start of a spontaneous breathing cycle.

In some examples, in the synchronous Nasal Intermittent Positive Pressure Ventilation mode, the apparatus is configured to provide gases to the patient at a set inspiratory airway pressure on detection of a start a spontaneous breathing cycle.

In some examples, in the synchronous Nasal Intermittent Positive Pressure Ventilation mode, the apparatus is configured to provide gases to the patient at a set expiratory airway pressure during expiration of the patient.

In some examples, in the synchronous Nasal Intermittent Positive Pressure Ventilation mode, the apparatus is configured to provide gases to the patient at a set inspiratory airway pressure during, at least part of, or all of inspiration of the patient.

In some examples, in the synchronous Nasal Intermittent Positive Pressure Ventilation mode, the apparatus is configured to provide gases to the patient at a set expiratory airway pressure at: a predetermined time after detection of a start a spontaneous breathing cycle, or at a predetermined time after detection of an end of inhalation of a spontaneous breathing cycle.

In some examples, in the synchronous Nasal Intermittent Positive Pressure Ventilation mode, a or the set inspiratory airway pressure is provided to the patient at a start of a spontaneous breath cycle, or after a predetermined time passes without detection of a start of a spontaneous breath cycle.

In some examples, the start of a spontaneous breath cycle is based on a determined flow exceeding a spontaneous breath flow rate threshold.

In some examples, the spontaneous breath flow rate threshold is about 0.5 Litres/minute to about 7.0 Litres/minutes.

In some examples, the predetermined time without detection of the start of a spontaneous breath cycle is based on a minimum breathing rate.

In some examples, in the synchronous Nasal Intermittent Positive Pressure Ventilation mode, the apparatus is configured to alternate in providing gases to the patient at a or the set inspiratory airway pressure during, and a the set expiratory airway pressure.

In some examples, a transition from providing the set expiratory airway pressure to the set inspiratory airway pressure is defined by a rise time.

In some examples, the rise time is about 0.1 seconds to about 2 seconds.

In some examples, a transition from providing the set inspiratory airway pressure to the set expiratory airway pressure is defined by a fall time.

In some examples, the fall time is about 0.1 seconds to about 2 seconds.

In some examples, the set inspiratory airway pressure is provided after the rise time for a latter portion of an inspiration time.

In some examples, the inspiration time is about 0.1 seconds to about 3 seconds.

In some examples, the inspiration time comprises the rise time and a time period for which the set inspiratory airway pressure is provided.

In some examples, the inspiration time is set so as not to exceed half of a period of a breath cycle.

In some examples, in the NIV-NAVA mode, the system is configured to control the flow and/or pressure of gas supplied to the patient based on the measurement of at least one physiological sensor attached to the patient.

In some examples, in the NIV-NAVA mode, the system comprises a physiological sensor.

In some examples, the physiological sensor comprises an electrical diaphragm sensor.

In some examples, the physiological sensor comprises an electromyography (EMG) sensor.

In some examples, the physiological sensor is in wired and/or wireless communication with the apparatus.

In some examples, the physiological sensor measures a patient characteristic.

In some examples, the patient characteristic comprises patient breathing.

In some examples, the physiological sensor is configured to measure a parameter indicative of patient inhalation and/or exhalation.

In some examples, the physiological sensor is configured to detect the start of patient inhalation and/or patient exhalation.

In some examples, the physiological sensor is configured to output signals which are in anticipation of patient inhalation and/or patient exhalation.

In some examples, in the NIV-NAVA mode, the patient is supplied with a flow of gas at a set inspiratory airway pressure during at least part of an inspiratory phase of a breathing cycle, and a set expiratory airway pressure during at least part of an expiratory phase of a breathing cycle.

In some examples, the delivery of the set inspiratory airway pressure and/or the set expiratory airway pressure may be based on a or the measured patient characteristic.

In some examples, in the NIV-NAVA mode, the apparatus delivers the flow of gases invasively.

In some examples, in the NIV-NAVA mode, the apparatus delivers the flow of gases non-invasively.

In some examples, in the NIV-NAVA mode, the system comprises an unsealed interface.

In some examples, in the NIV-NAVA mode, the system comprises a sealed interface.

In some examples, in the HFOV mode, the system is configured to deliver a set pressure level, with pressure oscillations superposed upon it.

In some examples, the frequency of the superposed pressure oscillations is 300 to 900 cycles per minute.

In some examples, in the HFOV mode, the system comprises an unsealed interface.

In some examples, in the HFOV mode, the system comprises a sealed interface.

In some examples, in the High Frequency Percussive Ventilation (HFPV) mode, the set base pressure level may vary between a first base pressure level and a second base pressure level.

In some examples, in the volume-limited pressure control mode, the system is configured to supply a patient with a flow of gas at a set pressure(s).

In some examples, in the volume-limited pressure control mode, the apparatus is configured to control the supply of the gas based on a delivered volume of gases to the patient.

In some examples, the apparatus is configured to monitor the delivered volume of gases to the patient.

In some examples, the apparatus is configured to compare the monitored delivered volume to a set volume range or threshold.

In some examples, the apparatus is configured to take a certain action if the monitored delivered volume is outside a or the set volume range and/or exceeds or is below a or the volume threshold.

In some examples, the certain action comprises decreasing the set pressure supplied to the patient.

In some examples, the certain action comprises increasing the set pressure supplied to the patient.

In some examples, the certain action comprises ceasing to provide pressure to the patient.

In some examples, the certain action comprises allowing exhalation.

In some examples, in the volume-limited pressure control mode, the apparatus is configured to control the supply of the gas based on the delivered volume during a breath.

In some examples, in the volume-limited pressure control mode, the system is configured to deliver invasive therapy.

In some examples, in the volume-limited pressure control mode, the system is configured to deliver non-invasive therapy.

In some examples, in the volume-limited pressure control mode, the system comprises a proximal pressure and/or a flow sensor at the patient end of the system.

In some examples, in the volume-limited pressure control mode, the system comprises an unsealed interface.

In some examples, in the volume-limited pressure control mode, the system comprises a sealed interface.

In some examples, in the volume controlled ventilation mode, the apparatus is configured to supply a patient with a flow of gas at a set volume per inhalation.

In some examples, the apparatus is configured to deliver the flow of gas at a set tidal volume and at a set flow rate.

In some examples, the apparatus is configured to control a or the flow rate and/or a or the pressure, to meet the set volume per inhalation.

In some examples, in the volume controlled ventilation mode, the apparatus comprises a set maximum flow rate that the apparatus may be unable to increase the flow of gases above.

In some examples, in the volume controlled ventilation mode, the system is configured to deliver invasive therapy.

In some examples, in the volume controlled ventilation mode, the system is configured to deliver non-invasive therapy.

In some examples, in the volume controlled ventilation mode, the system comprises a proximal pressure and/or a flow sensor at the patient end of the system.

In some examples, in the volume controlled ventilation mode, the system comprises an unsealed interface.

In some examples, in the volume controlled ventilation mode, the system comprises a sealed interface.

In some examples, in the resuscitation mode, the apparatus is configured to deliver a simulated breath to a patient for resuscitation purposes.

In some examples, the simulated breath is implemented by adjusting the set pressure of the apparatus between a first and second pressure.

In some examples, the pressure adjustment is achieved by a trigger of the respiratory therapy system.

In some examples, a user or clinician may interact with the trigger to adjust the pressure between the first and the second pressures.

In some examples, the trigger is located on a component of the system.

In some examples, in the resuscitation mode, the system comprises an unsealed interface.

In some examples, in the resuscitation mode, the system comprises a sealed interface.

In some examples, in a or the variable flow CPAP therapy mode the one or more alarms comprise:
a) An over pressure alarm,
b) An elevated pressure alarm,
c) A low pressure alarm,
d) An excessive leak alarm,
e) A blockage alarm,
f) An apnea alarm,
g) Any combination of a)-f).

In some examples, in a or the synchronous Nasal Intermittent Positive Pressure Ventilation therapy mode and/or the asynchronous Nasal Intermittent Positive Pressure Ventilation therapy mode the one or more alarms comprise:
a) An over pressure alarm,
b) An elevated pressure alarm,
c) A low pressure alarm,
d) An excessive leak alarm,
e) A blockage alarm,
f) An apnea alarm,
g) Any combination of a)-f).

In some examples, in a or the bubble CPAP mode, the one more alarms comprise an or the intermittent or no bubbling alarm.

In some examples, in a or the NIV-NAVA mode, the one or more alarms comprise a or the loss of physiological signal alarm.

In some examples, in a or the volume-limited pressure control mode, the one or more alarms comprise a or the tidal volume alarm.

In some examples, in a or the high flow mode, the one or more alarms may comprise a or the prong dislodge alarm.

In some examples, the variable flow CPAP mode, the synchronous Nasal Intermittent Positive Pressure Ventilation therapy mode and the asynchronous Nasal Intermittent Positive Pressure Ventilation therapy mode have an or the associated over pressure alarm.

In some examples, the over pressure alarm is generated when a or the determined pressure is greater than an over pressure threshold.

In some examples, a or the over pressure alarm is generated when the determined pressure is greater than an over pressure threshold for an over pressure time period.

In some examples, the over pressure time period is about 1 second.

In some examples, the over pressure threshold for the over pressure alarm associated with the variable flow CPAP mode is about 50 cmH2O to about 60 cmH2O, or about 55 cmH2O.

In some examples, the over pressure threshold is settable by a user.

In some examples, when the over pressure alarm is generated, the controller is configured to:
a) Disable the flow generator,
b) Disable a heater of the humidifier,
c) Disable a heater of an attached conduit,
d) Display a message on a display,
e) Any combination of a)-d).

In some examples, the variable flow CPAP mode, the synchronous Nasal Intermittent Positive Pressure Ventilation therapy mode and the asynchronous Nasal Intermittent Positive Pressure Ventilation therapy mode have an associated elevated pressure alarm.

In some examples, a or the elevated pressure alarm is generated when a or the determined pressure is greater than an elevated pressure threshold associated with one or more of the plurality of therapy modes.

In some examples, the elevated pressure alarm is generated when the determined pressure is greater than the elevated pressure threshold for an elevated pressure time period.

In some examples, the elevated pressure time period is greater than an or the over pressure time period.

In some examples, the elevated pressure time period is about 5 seconds.

In some examples, the elevated pressure threshold is lower than a or the over pressure threshold.

In some examples, in the variable flow CPAP mode, the elevated pressure threshold is based on a or the set pressure of the variable flow CPAP mode.

In some examples, in the variable flow CPAP mode, the elevated pressure threshold is greater than a or the set pressure of the variable flow CPAP mode by about 3 cmH2O to about 7 cmH2O, or about 5 cmH2O.

In some examples, in the synchronous Nasal Intermittent Positive Pressure Ventilation mode and/or the asynchronous Nasal Intermittent Positive Pressure Ventilation mode, the elevated pressure threshold is greater than a or the set inspiratory airway pressure.

In some examples, in the synchronous Nasal Intermittent Positive Pressure Ventilation mode and/or the asynchronous Nasal Intermittent Positive Pressure Ventilation mode, the elevated pressure threshold is greater than a or the set inspiratory airway pressure by about 3 cmH2O to about 7 cmH2O, or about 5 cmH2O.

In some examples, when the elevated pressure alarm is generated, the controller is configured to:
a) Disable the flow generator,
b) Disable a heater of the humidifier,
c) Disable a heater of an attached conduit,
d) Display a message on a display,
e) Generate an audio warning
f) Generate a visual warning
g) Any combination of a)-f).

In some examples, the variable flow CPAP mode, the synchronous Nasal Intermittent Positive Pressure Ventilation therapy mode and the asynchronous Nasal Intermittent Positive Pressure Ventilation therapy mode have an or the associated low pressure alarm.

In some examples, a or the low pressure alarm is generated when a or the determined pressure is less than an low pressure threshold associated with one or more of the plurality of therapy modes.

In some examples, the low pressure alarm is generated when the determined pressure is lower than the low pressure threshold for a low pressure time period.

In some examples, the low pressure time period is about 10 seconds.

In some examples, in the variable flow CPAP mode, the low pressure threshold is based on a or the set pressure of the variable flow CPAP mode.

In some examples, in the variable flow CPAP mode, the elevated pressure threshold is less than a or the set pressure of the variable flow CPAP mode by about 1 cmH2O to about 3 cmH2O, or about 1 cmH2O.

In some examples, in the synchronous Nasal Intermittent Positive Pressure Ventilation mode and/or the asynchronous Nasal Intermittent Positive Pressure Ventilation mode, the low pressure threshold is less than a or the set expiratory airway pressure.

In some examples, in the synchronous Nasal Intermittent Positive Pressure Ventilation mode and/or the asynchronous Nasal Intermittent Positive Pressure Ventilation mode, the low pressure threshold is less than a or the set expiratory airway pressure by about 1 cmH2O to about 3 cmH2O, or about 1 cmH2O.

In some examples, when the low pressure alarm is generated, the controller is configured to:
a) Disable a heater of the humidifier,
b) Disable a heater of an attached conduit,
c) Display a message on a display,
d) Any combination of a)-c).

In some examples, a or the excessive leak alarm is generated when a or the determined flow is greater than an excessive leak threshold associated with one or more of the plurality of therapy modes.

In some examples, a or the excessive leak alarm associated is generated when the determined flow rate is greater than an excessive leak threshold for an excessive leak time period.

In some examples, the excessive time period is about 10 seconds.

In some examples, the excessive leak threshold is based on a or the determined pressure.

In some examples, the determined pressure in the gases flow path is a pressure at or near the patient and/or at an end of an inspiratory conduit.

In some examples, the excessive leak threshold is based on a user settable variable indicative of an acceptable unintentional leak flow rate.

In some examples, the acceptable unintentional leak flow rate is based on an allowed leak flow rate at a particular pressure.

In some examples, the excessive leak threshold is based a flow through the pressure regulator and a flow caused by leak.

In some examples, the flow through the pressure regulator is based on a conductance of the pressure regulator and a set pressure.

In some examples, the flow caused by leak is a function of the allowed leak set by the user and the particular pressure.

In some examples, the excessive leak alarm is cleared when a or the determined flow rate is lower than the excessive leak threshold.

In some examples, the excessive leak alarm is cleared when the determined flow rate is lower than the excessive leak threshold, for an excessive leak time clear period.

In some examples, the excessive leak time clear period about 3 seconds.

In some examples, once the excessive leak alarm has been generated, the alarm cannot be cleared for a predetermined time, wherein the predetermined time is about 5 seconds.

In some examples, when the excessive leak alarm is generated, the controller is configured to:
a) Display a message on a display,
b) Generate an audio warning
c) Generate a visual warning
d) Any combination of a)-c).

In some examples, a or the prong dislodge alarm is be generated when a prong dislodgement leak is detected.

In some examples, the apparatus is configured to recognize a flow rate over a prong dislodgement threshold for a given pressure as being indicative of a prong dislodgement.

In some examples, the prong dislodge alarm is only generated when the measured flow rate increases over the prong dislodgement threshold for a predetermined period.

In some examples, in response to the prong dislodge alarm being generated, the apparatus transitions to a flow control mode.

In some examples, the prong dislodge alarm is configured to be stopped manually by a user and/or stopped automatically and/or stopped semi-automatically.

In some examples, a or the blockage alarm is generated when the determined flow is less than an blockage threshold associated with one or more of the plurality of therapy modes.

In some examples, a or the blockage alarm is generated when the determined flow rate is less than a blockage threshold for a blockage time period.

In some examples, the blockage time period is about 10 seconds.

In some examples, the blockage threshold is based on a or the determined pressure.

In some examples, the determined pressure in the gases flow path is a pressure at or near the patient and/or at a patient end of an inspiratory conduit.

In some examples, the blockage alarm is cleared when the determined flow rate is lower than a blockage threshold.

In some examples, the blockage alarm is cleared when the determined flow rate is lower than a blockage threshold for a blockage time clear period.

In some examples, the blockage time clear period about 10 seconds.

In some examples, when the blockage alarm is generated, the controller is configured to:
a) Disable a heater of the humidifier,
b) Disable a heater of an attached conduit,
c) Display a message on a display,
d) Any combination of a)-c).

In some examples, a or the apnea alarm is triggered when there are no breathing cycles detected.

In some examples, in the asynchronous Nasal Intermittent Positive Pressure Ventilation mode, a or the apnea alarm is triggered when no breathing cycles are provided to the patient.

In some examples, the apnea alarm is triggered when there are no breathing cycles detected, within an apnea time period.

In some examples, the apnea time period is about 5 seconds to about 60 seconds.

In some examples, the breathing cycle is detected based on the detection of a start of a spontaneous breathing cycle and/or in asynchronous Nasal Intermittent Positive Pressure Ventilation mode when a breathing cycle is provided to the patient to cause inspiration and expiration.

In some examples, breathing cycles are detected based on a determined flow and/or a determined pressure.

In some examples, breathing cycles are detected based on detection of a positive peak followed by a negative peak of the determined flow and/or a determined pressure.

In some examples, breathing cycles are detected based on detection of a positive peak followed by a negative peak, wherein the difference between the amplitude of the positive peak and the negative peak is greater than a breath amplitude threshold.

In some examples, the breath amplitude threshold is about 5 to about 200 mL.

In some examples, the apnea alarm is cleared when a breathing cycle is detected.

In some examples, once the apnea alarm has been generated, the alarm cannot be cleared for a predetermined time, wherein the predetermined time is about 5 seconds.

In some examples, when the apnea alarm is generated, the controller is configured to:
a) Display a message on a display,
b) Generate an audio warning
c) Generate a visual warning
d) Any combination of a)-c).

In some examples, a or the intermittent or no bubbling alarm is generated when bubbling is intermittent and/or has ceased.

In some examples, bubbling is measured by one or more sensors in the system.

In some examples, the intermittent or no bubbling alarm is generated when a variable indicative of bubbling falls below a threshold.

In some examples, the intermittent or no bubbling alarm is generated when the variable indicative of bubbling falls below a bubbling threshold for a predetermined period.

In some examples, the intermittent or no bubbling alarm is configured to be stopped manually by a user and/or stopped automatically and/or stopped semi-automatically.

In some examples, a or the condensation alarm is generated when condensation is detected in the system.

In some examples, the breathing assistance system and/or apparatus comprises a condensation detector configured to measure condensation.

In some examples, the condensation alarm is generated when the measured condensation increases above a condensation threshold.

In some examples, the condensation alarm is generated when the measured condensation is above a condensation threshold for a predetermined period.

In some examples, the condensation alarm is configured to be stopped manually by a user and/or stopped automatically and/or stopped semi-automatically.

In some examples, a or the incorrect hardware alarm is generated when connection of incorrect hardware is detected.

In some examples, the incorrect hardware alarm is generated based on a comparison of the measured operational parameters of the connected hardware, with the known operational parameters of the correct hardware.

In some examples the operational parameters comprise flow and/or pressure characteristics.

In some examples, the system is configured to be non-operational whilst the incorrect hardware alarm has been activated.

In some examples, the alarm is stopped by the removal of the incorrect hardware.

In some examples, a or the loss of physiological signal alarm is generated when the connection between a physiological sensor and the apparatus has been lost.

In some examples, the patient is equipped with at least one physiological sensor.

In some examples, the loss of physiological signal alarm is configured to be stopped manually, and/or stopped semi-automatically, and/or stopped automatically.

In some examples, the system comprises at least one parameter sensor(s).

In some examples, the at least one parameter sensor(s) are configured to directly or indirectly measure a signal indicative of a patient parameter.

In some examples, the at least one parameter sensor(s) comprise a breath rate sensor, a tidal volume sensor, a SpO2 sensor, and/or an exhaled CO2 sensor.

In some examples, the breath rate sensor is configured to measure breath rate.

In some examples, the tidal volume sensor measures tidal volume.

In some examples, the SpO2 sensor measures the SpO2 of the patient.

In some examples, the exhaled CO2 sensor measures the amount of CO2 exhaled by the patient.

In some examples, the apparatus is configured to make adjustments to therapy parameters based on the measurements of the at least one parameter sensor(s).

In some examples, a or the high/low breath rate alarm is generated when a or the measured breath rate is considered too high or too low.

In some examples, the high/low breath rate alarm is generated when the system determines the measured breath rate is above a high breath rate threshold or below a low breath rate threshold. In some examples, the high/low breath rate alarm is generated when the measured breath rate is above/below the high/low breath thresholds for a predetermined period.

In some examples, the high/low breath rate alarm is configured to be stopped manually by a user and/or stopped automatically and/or stopped semi-automatically.

In some examples, a or the tidal volume alarm is generated when the tidal volume increases or decreases out of a safe range.

In some examples, the safe range is defined by a lower and/or upper tidal volume threshold.

In some examples, the tidal volume is based on measurements made by a or the tidal volume sensor(s).

In some examples, the tidal volume alarm is configured to be stopped manually by a user and/or stopped automatically and/or semi-automatically.

In some examples, a or the SpO2 alarm is generated when the measured SpO2 is considered too high or too low.

In some examples, the SpO2 alarm is generated when the measured SpO2 is above a high SpO2 threshold or below a low SpO2 threshold.

In some examples, the SpO2 alarm is generated when the measured SpO2 is above/below the SpO2 thresholds for a predetermined period.

In some examples, the SpO2 alarm is configured to be stopped manually by a user and/or stopped automatically and/or stopped semi-automatically.

In some examples, a or the exhaled CO2 alarm is generated when the measured exhaled CO2 is considered too high.

In some examples, the exhaled CO2 alarm is generated when the measured exhaled CO2 levels is above an exhaled CO2 threshold.

In some examples, the exhaled CO2 alarm is generated when the measured exhaled CO2 is above the exhaled CO2 threshold for a predetermined period.

In some examples, the exhaled CO2 alarm is configured to be stopped manually by a user and/or stopped automatically and/or stopped semi-automatically.

In another aspect there is disclosed a breathing assistance apparatus configured to deliver respiratory therapy to a patient, the breathing assistance apparatus comprising:
a flow generator, and a humidifier in fluid communication with the flow generator,
a controller configured to control the breathing assistance apparatus,
wherein the breathing assistance apparatus is changeable between a plurality of therapy modes, wherein the plurality of therapy modes comprise: a high flow therapy mode, a Bubble CPAP therapy mode, a variable flow CPAP mode, an asynchronous Nasal Intermittent Positive Pressure Ventilation mode and a synchronous Nasal Intermittent Positive Pressure Ventilation mode, a Non-Invasive Ventilation Neurally Adjusted Ventilatory Assist (NIV-NAVA) mode, a High Frequency Oscillatory Ventilation (HFOV) mode, a volume-limited pressure control mode, a volume control ventilation mode, and a resuscitation mode
wherein:
   in the high flow therapy mode the breathing assistance apparatus is configured to provide high flow therapy,
   in the Bubble CPAP therapy mode the breathing assistance apparatus is configured to provide bubble CPAP therapy,
   in the variable flow CPAP mode the breathing assistance apparatus is configured to provide variable flow CPAP therapy,
   in the asynchronous Nasal Intermittent Positive Pressure Ventilation mode the breathing assistance apparatus is configured to provide asynchronous Nasal Intermittent Positive Pressure Ventilation therapy,
   in the synchronous Nasal Intermittent Positive Pressure Ventilation mode the breathing assistance apparatus is configured to provide synchronous Nasal Intermittent Positive Pressure Ventilation therapy,
   in the NIV-NAVA mode the breathing assistance apparatus is configured to provide NIV-NAVA therapy,
   in the HFOV mode the breathing assistance apparatus is configured to provide HFOV therapy,
   in the volume-limited pressure control mode the breathing assistance apparatus is configured to provide volume-limited pressure control therapy,
   in the volume control ventilation mode, the breathing assistance apparatus is configured to provide volume control ventilation therapy, and
   in the resuscitation mode the breathing assistance apparatus is configured to provide resuscitation therapy.

In some examples, the plurality of therapy modes further comprises High Frequency Percussive Ventilation (HFPV) mode, in which the breathing assistance apparatus is configured to provide HFPV therapy.

In an aspect there is disclosed a breathing assistance apparatus configured to deliver respiratory therapy to a patient, the breathing assistance apparatus comprising:
a flow generator, and a humidifier in fluid communication with the flow generator,
a controller configured to control the breathing assistance apparatus,
wherein the breathing assistance apparatus is configured to operate in one or more therapy modes.

In some examples, the controller is configured to generate one or more alarms.

In some examples, in one or any combination of: the variable flow CPAP mode, the asynchronous Nasal Intermittent Positive Pressure Ventilation mode and/or the synchronous Nasal Intermittent Positive Pressure Ventilation mode; the controller is configured to control the flow generator according to pressure control, wherein pressure control comprises controlling the flow generator to provide the flow of gases at a set pressure, or according to flow control, wherein flow control comprises controlling the flow generator to provide the flow of gases at a set flow rate, the controller configured to:
control the flow generator according to flow control if a determined flow rate is greater than a flow rate threshold, and
control the flow generator according to pressure control if the determined flow rate is lower than the flow rate threshold.

In some examples, the controller is configured to control the flow generator according to pressure control, wherein pressure control comprises controlling the flow generator to provide the flow of gases at a set pressure, or according to flow control, wherein flow control comprises controlling the flow generator to provide the flow of gases at a set flow rate, the controller configured to:
control the flow generator according to flow control if a determined flow rate is greater than a flow rate threshold, and
control the flow generator according to pressure control if the determined flow rate is lower than the flow rate threshold.

In another aspect there is disclosed a breathing assistance apparatus for providing respiratory therapy, the breathing assistance apparatus comprising:
a flow generator, the flow generator configured to provide a flow of gases to an inspiratory conduit,
a controller configured to be in communication with at least one gases property sensor, wherein the controller is configured to calculate a determined flow rate and a determined pressure of the gases in a gases flow pathway based on an least an output of the at least one gases property sensor,
wherein the gases flow pathway comprises at least the inspiratory conduit configured to be connected to a patient interface, and an expiratory conduit configured to be connected to a patient interface and a pressure regulator, the pressure regulator comprising an expiratory orifice,
the controller is configured to control the flow generator according to pressure control, wherein pressure control comprises controlling the flow generator to provide the flow of gases at a set pressure, or according to flow control, wherein flow control comprises controlling the flow generator to provide the flow of gases at a set flow rate, the controller configured to:
   control the flow generator according to flow control if a determined flow rate is greater than a flow rate threshold, and
   control the flow generator according to pressure control if the determined flow rate is lower than the flow rate threshold.

In some examples, the set flow rate is a function of the set pressure.

In some examples, the set flow rate of is a function of the set pressure and a conductance of the pressure regulator, and a conductance of the unintentional leak.

In some examples, the set flow rate is a maximum allowed flow rate for the set pressure.

In some examples, the set flow rate provides pressure support where leak cannot be compensated for when the controller controls the flow generator to provide the flow of gases at a set pressure.

In some examples, the flow rate threshold is a maximum allowed flow rate for the system for the determined pressure.

In some examples, the maximum allowed flow rate is the sum of at least: a flow through a pressure regulator, and a flow from unintentional leak.

In some examples, the flow through the pressure regulator is a function of a conductance of the pressure regulator and a determined pressure.

In some examples, the flow from unintentional leak is a function of a conductance of the unintentional leak and a or the determined pressure.

In some examples, the determined pressure is a pressure at or near the patient end of the or an inspiratory conduit.

In some examples, the flow rate threshold is dynamic.

In some examples, the flow rate threshold is continuously compared to the determined flow rate.

In some examples, the controller is configured to control the flow generator according to pressure control when the determined flow rate is lower than the flow rate threshold for a time period.

In some examples, the time period is about 0.3 seconds to about 1 second or about 0.3 seconds

In some examples, the controller is configured to control the flow generator according to flow control when the determined flow rate is greater than the flow rate threshold for a time period.

In some examples, the time period is about 0.3 seconds to about 1 second or about 0.3 seconds

In some examples, on commencement of therapy the apparatus is configured to control the flow generator according to pressure control.

In some examples, the set pressure is provided by a user via a user interface.

In some examples, the apparatus comprising a supplemental gases inlet configured to receive a supplemental gas and an ambient inlet to receive ambient air.

In some examples, the supplemental gas inlet is separate from the ambient inlet.

In some examples, the supplemental gas inlet is an oxygen inlet, and the supplemental gas is oxygen.

In some examples, the supplemental gas and ambient air are mixed in a gases mixer.

In some examples, the gases mixer is upstream, or downstream, of the flow generator.

In some examples, the gases mixer is part of the apparatus.

In some examples, a blower of the flow generator is configured to mix ambient air from the ambient air inlet and supplemental gas from the supplemental gas inlet.

In some examples, the controller is further configured to control the amount of supplemental gas in the flow of gases by controlling opening of a supplemental gas inlet valve.

In some examples, the controller is further configured to control the amount of supplemental gas in the flow of gases based on an output of a pulse oximeter.

In some examples, the controller is further configured to control the amount of supplemental gas in the flow of gases based on an output of an oxygen concentration sensor.

In some examples, apparatus comprises a battery.

In some examples, the battery is a primary source, or an auxiliary source of power for the apparatus.

In some examples, the battery is the source of power for the apparatus when a mains power supply is not connected.

In some examples, the at least one gases property sensor is located at one or more of:
a) in the breathing assistance apparatus, optionally within the flow generator,
b) in a patient interface,
c) in a pressure regulator,
d) in a or the inspiratory conduit and/or expiratory conduit
e) Any combination of a)-d).

In some examples, the determined flow rate and/or determined pressure is an estimate of a flow rate and/or pressure at a location in the gases flow path.

In some examples, the location in the gases flow path is the same or different to where the at least one gases property sensor is located.

In some examples, the determined pressure is an estimate of a pressure at or near the patient in the gases flow path and/or at the end of an inspiratory conduit.

In some examples, the determined pressure is indicative of a pressure at the end of the inspiratory conduit, and wherein the determined pressure is based on the output of the gases property sensor indicative of a flow rate of the gases in a gases flow pathway.

In some examples, the determined pressure is indicative of a pressure at the end of the inspiratory conduit, and wherein the determined pressure is based on a characteristic of the inspiratory conduit,

In some examples, the at least one gases property sensor comprises a flow rate sensor and/or a pressure sensor.

In some examples, the at least one gases property sensor is part of the apparatus.

In some examples, the at least one gases property sensor is external, and connectable to the apparatus.

In some examples, the at least one gases property sensor is connectable to the apparatus via a wired connection and/or a wireless connection.

In some examples, the inspiratory conduit and/or expiratory conduit comprises a port, and wherein the at least one gases property sensor is insertable into the port to be in contact with the gases in the gases flow path.

In some examples, the at least one gases property sensor comprises an oxygen concentration sensor, wherein the controller is configured to calculate an oxygen concentration of the of the gases in a gases flow pathway based on at least an output of the at least one gases property sensor.

In some examples, the apparatus comprises a housing, and wherein the housing is configured to house:
a) a blower of the flow generator,
b) a humidifier,
c) the controller of the apparatus,
d) an oxygen sensor,
e) a gases mixer,
f) a battery,
g) a supplemental gases inlet,
h) an ambient air outlet,
i) any combination of a)-h).

In some examples, the apparatus is configured to connect to a pulse oximeter, wherein the controller is configured to calculate a patient's oxygen saturation based on at least an output of the pulse oximeter.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 illustrates schematically a conventional setup of using a breathing assistance apparatus to provide bubble CPAP.

FIG. 2 illustrates schematically a respiratory system to provide bubble CPAP.

FIG. 3 illustrates schematically a respiratory system to provide variable flow CPAP.

FIG. 4 illustrates schematically a respiratory system to provide asynchronous Nasal Intermittent Positive Pressure Ventilation and synchronous Nasal Intermittent Positive Pressure Ventilation therapy.

FIG. 5 illustrates schematically a high flow respiratory system configured to provide a respiratory therapy to a patient.

FIG. 6A is a front perspective view of an example high flow breathing assistance apparatus with a humidification chamber in position.

FIG. 6B is a back perspective view of the breathing assistance apparatus of FIG. 6A.

FIG. 6C illustrates an example sensing chamber of the breathing assistance apparatus of FIG. 6A.

FIG. 7 illustrates a schematic view of an apparatus.

FIG. 8 illustrates the pressure provided during Nasal Intermittent Positive Pressure Ventilation therapy.

FIGS. 9 and 9A illustrate a breathing assistance apparatus having a various control programs relating to therapy modes.

FIG. 10 illustrates an example of various pressure based alarms.

FIG. 11 illustrates a control scheme for variable flow CPAP.

### DETAILED DESCRIPTION

Although certain examples are described below, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below.

Various respiratory therapies may need to be provided in a hospital setting. If for example a patient's condition changes and a change of therapy is required a clinician may have to utilize a new apparatus with different components which is capable of providing the new therapy. This may be inconvenient for the clinician, may take the clinician an extended amount of time. In some cases, there may be a delay in changing therapy until a clinician has the required time to locate the new apparatus and set it up.

The disclosure provides for a breathing assistance apparatus which is configurable to provide for a number of different respiratory therapies. This may allow for escalation and de-escalation between different therapies for the patient. It may also allow for changing between different therapies depending on patient condition.

The apparatus of the disclosure may be particularly applicable to neonatal patients, and the therapy modes provided be directed to treatment of neonatal patients. In some cases, the apparatus may be applicable to pediatric patients, and the therapy modes provided be directed to treatment of pediatric patients.

The apparatus may be for treatment of neonates and/or infant and/or pediatric patients. As described in detail below the therapies provided may be at flow or pressure ranges suitable for use with neonates and/or infant and/or pediatric patients.

The apparatus may be configured to provide: high flow therapy, Bubble CPAP therapy, variable flow CPAP, asynchronous Nasal Intermittent Positive Pressure Ventilation, synchronous Nasal Intermittent Positive Pressure Ventilation, Non-Invasive Ventilation Neurally Adjusted Ventilatory Assist (NIV-NAVA) therapy, High Frequency Oscillatory Ventilation (HFOV) therapy, volume-limited pressure control therapy, volume control ventilation therapy, and resuscitation therapy. These therapies will be discussed in more detail below.

An apparatus which contains both a flow generator and a humidifier may also decrease set up complexity and decrease any required reconfiguration when changing therapies. An apparatus which contains both a flow generator and a humidifier may also increase usability.

The term therapy in the specification may be used to also include types of respiratory support or respiratory assistance.

Different therapies may require different alarms. Disclosed herein is an apparatus which comprises different alarms for different therapy types. This may improve safety of the therapies and minimize the risk of misconfiguration of the apparatus when the therapy type is changed.

High flow therapy as discussed herein is intended to be given its typical ordinary meaning as understood by a person of skill in the art which generally refers to a respiratory assistance system delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute to about sixty liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), or high flow therapy (HFT) among other common names.

Bubble Continuous Positive Airway Pressure (CPAP) is a form of respiratory therapy in which a patient (typically an infant) is supplied with a flow of gas via a patient interface. The flow of gas may be provided by the apparatus as disclosed herein provided by a gas source in the wall of a hospital or clinic, or may be provided by cylinders of compressed air and/or oxygen, for example during transport. The patient interface is connected to two conduits, which are an inspiratory conduit and an expiratory conduit. The inspiratory conduit provides gas to the patient. The expiratory conduit provides a passage for exhaled gases from the patient. The expiratory conduit is in communication with a pressure regulator, which is used to set pressure. The pressure regulator may be a chamber with a column of water into which an end portion of the expiratory conduit is submerged. The exhaled gases are discharged into the pressure regulator. The exhaled gases being discharged into the water results in bubbling of the water i.e. a bubbling effect. The patient interface is typically configured to form a seal with the patient's mouth and/or nose. Examples of sealed patient interfaces can include a nasal mask, an oral mask, a full face mask, nasal pillows, or a cannula with sealing nasal prongs.

Bubble CPAP therapy can produce variations or oscillations in the pressure of gases supplied to a patient connected to a positive pressure ventilation device. By submerging one end of the expiratory conduit into a water column, the resulting bubbles generate a variation or ripple in the pressure of gases delivered to the patient. The bubble CPAP system also provides a method of varying a mean pressure of gases supplied to the patient by variation of the level to which the end of the expiratory conduit is submerged within the water column. The level of submergence of the end of the expiratory conduit can be kept constant in order to maintain the mean pressure of gases supplied to the patient.

In some configurations there may be multiple bubble CPAP modes where the pressure oscillations may be provided in different ways. In some examples the various the bubble CPAP modes where the pressure oscillations may be provided in different ways may be separate modes to bubble CPAP mode, or sub-modes of bubble CPAP mode.

Variable flow CPAP is a form of respiratory therapy in which a patient (typically an infant) is supplied with a flow of gas at a set pressure via a patient interface. Variable flow CPAP may also be known as single limb CPAP.

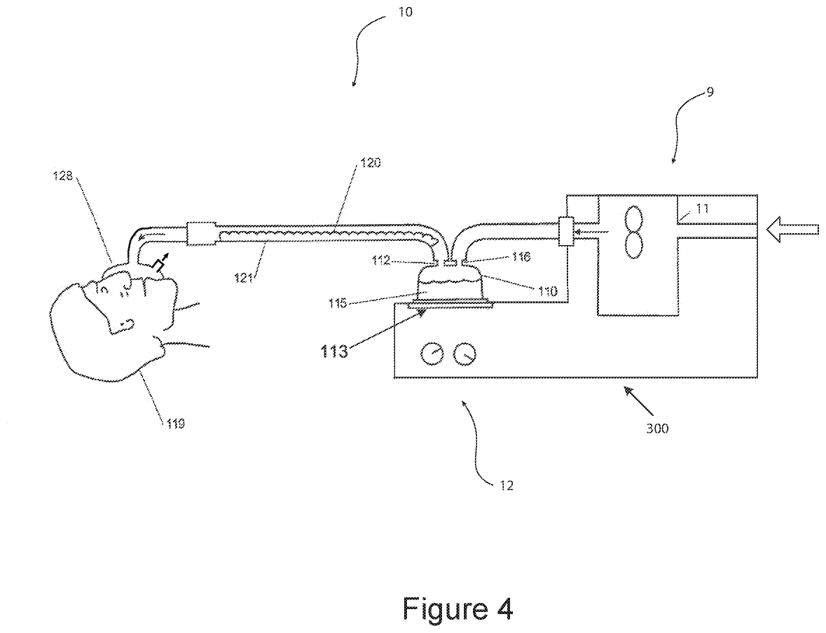

The flow of gas may be provided by the apparatus as disclosed. The patient interface is connected to two conduits, which are an inspiratory conduit and an expiratory conduit. The inspiratory conduit provides gas to the patient. The expiratory conduit provides a passage for exhaled gases from the patient. The expiratory conduit is in communication with a pressure regulator. The pressure regulator may be an expiratory orifice which is configured to provide for a flow restriction. In contrast to bubble CPAP where the flow rate of the delivered gases is constant, the pressure of the delivered gases is controlled (and the flow rate is variable). In some examples, the expiratory orifice may be provided elsewhere in the system, for example as part of the interface as shown min FIG. 4.

Nasal Intermittent Positive Pressure Ventilation is a form of respiratory therapy in which a patient (typically an infant) is supplied with a flow of gas at a set inspiratory airway pressure during at least part of an inspiratory phase of a breathing cycle, and a set expiratory airway pressure during at least part of an expiratory phase of a breathing cycle. In some cases the set expiratory airway pressure may be chosen in a similar way to Bubble CPAP therapy.

There may be two types of Nasal Intermittent Positive Pressure Ventilation asynchronous and synchronous.

In asynchronous Nasal Intermittent Positive Pressure Ventilation provision of the set inspiratory airway pressure and expiratory airway pressure is independent of any spontaneous breathing of the patient.

In synchronous Nasal Intermittent Positive Pressure Ventilation provision of the set inspiratory airway pressure may be synchronized with at least the start of a breathing cycle (i.e. the start of inhalation). In other words, the provision of inspiratory airway pressure may be triggered by the start of a breathing cycle.

Non-Invasive Ventilation Neurally Adjusted Ventilatory Assist (NIV-NAVA) therapy is a form of respiratory therapy in which a patient is supplied with a flow of gas based on the measurement of at least one physiological sensor attached to a patient. The at least one physiological sensor may measure a patient characteristic such as patient breathing. The patient may be supplied with a flow of gas at a set inspiratory airway pressure during at least part of an inspiratory phase of a breathing cycle, and a set expiratory airway pressure during at least part of an expiratory phase of a breathing cycle. The delivery of the set inspiratory airway pressure and/or set expiratory airway pressure may be based on the measured patient characteristic (such as patient breathing). In some configurations, the set inspiratory airway pressure may be synchronized with at least the start of a breathing cycle (i.e. the start of inhalation). In other words, the provision of inspiratory airway pressure may be triggered by the start of a breathing cycle for example as in synchronous Nasal Intermittent Positive Pressure Ventilation. NIV-NAVA therapy may provide a benefit compared to other therapies that utilize apparatus sensors (such as Nasal Intermittent Positive Pressure Ventilation), as these systems detect inhalation or exhalation after it has already begun, as the apparatus sensors detect the result of inhalation or exhalation of the patient. In NIV-NAVA therapy, physiological sensors are used, which produce a signal indicative of inhalation and/or exhalation, so that inhalation and/or exhalation may be detected earlier and/or anticipated before they happen. In some configurations, the sensor may measure electrical activity at the diaphragm of a patient. Electrical activity of the diaphragm may precede changes in flow and/or pressure that occur as the patient starts inspiration, and so may provide a signal to the apparatus of patient breathing earlier than signals based on flow and/or pressure. Appropriate physiological sensors may comprise electrical diaphragm sensors and/or electromyography (EMG) sensors. In other configurations, the sensor may comprise an electroencephalography (EEG) sensor(s), to detect a neural signal going from the brain to send the electrical signal to the diaphragm.

High Frequency Oscillatory Ventilation (HFOV) therapy is a form of respiratory therapy in which a patient is supplied with a flow of gas at a set base pressure level with frequency oscillation superposed upon the base pressure level. In some configurations the set base pressure level may vary between a first base pressure level and a second base pressure level. In some configurations the first base pressure level may be an inspiratory airway pressure and may be delivered during at least part of an inspiratory phase of a breathing cycle; the second base pressure level may be expiratory airway pressure and may be delivered during at least part of an expiratory phase of a breathing cycle. HFOV therapy may have various clinical benefits. For instance, HFOV therapy may achieve or promote gas exchange with relatively smaller pressure changes and volumes compared to conventional ventilation. HFOV therapy may be for instance invasive or non-invasive. The very high frequency oscillations may be achieved by various techniques known by persons skilled in the art. For instance, they may be achieved using a valving arrangement and/or by applying a vibrating speaker.

Volume-limited pressure control is a form of respiratory therapy in which a patient is supplied with a flow of gas, but the supply is regulated based on a delivered volume of gases to the patient during inhalation. The patient is supplied with a flow of gas at a set inspiratory airway pressure during at least part of an inspiratory phase of a breathing cycle, and a set expiratory airway pressure during at least part of an expiratory phase of a breathing cycle (which may be for example a positive expiratory pressure). In some configurations the set inspiratory airway pressure may be the same as the set expiratory airway pressure (i.e. there is no change in set pressure between inspiration and expiration). There may be a maximum pressure limit set by a clinician. During provision of the flow during inhalation, the delivered volume to the patient may be monitored. The apparatus may control the supply of the gas based on the delivered volume. Delivered volume may be determined based on the output of one or more sensors (for example flow and/or pressure sensors). In some examples, the delivered volume may be based on the integral of the flow rate of the gases over inspiration.

In some examples, delivered volume may be determined by Respiratory inductance plethysmography. Respiratory inductance plethysmography uses sensors which measure chest movements to derive the volume of gases for example of an inhalation and/or exhalation.

In some configurations if the delivered volume reaches or exceeds a certain maximum volume threshold, then the apparatus may take a certain action. The action may for instance be to allow exhalation and/or to cease providing pressure to the patient and/or decrease the set inspiratory airway pressure.

In some configurations if the delivered volume is below a minimum volume threshold, then the apparatus may take a certain action. The action may for instance be to increase the set inspiratory airway pressure which may increase the volume of gases delivered to the patient. The action may be taken on the same breath or a following breath.

The apparatus may also change the set inspiratory airway pressure based on the delivered volume during a breath. For example, if an estimated delivered volume is less than a minimum volume threshold then the apparatus may increase the set inspiratory airway pressure so as to target the delivered volume to the minimum volume threshold. Additionally, or alternatively, if an estimated delivered volume is greater than a maximum volume threshold then the apparatus may decrease the set inspiratory airway pressure so as to target the delivered volume to the maximum volume threshold. The estimated volume may be based on the flow rate of the gases provided to the patient and the time of inspiration. In some examples, the estimated volume may be based on the integral of the flow rate of the gases provided to the patient for an expected duration of inspiration. The expected duration of inspiration may be based on historical data of previous breathing cycles.

In some examples, the estimated volume may be based on the delivered volume part way through the breathing cycle. For example, the estimated volume may be the sum of delivered volume so far in an inhalation and an estimate of how much volume is still to be provided.

Volume limiting the delivery of therapy may be beneficial as it may ensure that patients receive therapy within a safe tidal volume range. Volume-limited pressure control may be invasive or non-invasive. In some configurations (for example in non-invasive configurations) the system may preferably comprise a proximal pressure and/or flow sensor at the patient interface end of the system, thereby enabling non-invasive volume-based control. However, it will be appreciated that an invasive configuration may include such sensors.

Volume controlled ventilation is a form of respiratory therapy in which a patient is supplied with a flow of gas at a set volume per inhalation. The apparatus may control a flow rate and/or pressure, to meet this set volume. For example, the apparatus may increase or decrease flow rate and/or pressure (for example set inspiratory pressure) to achieve the set volume. The flow rate and/or pressure may be controlled synchronously, or asynchronously with respect to the patient's breathing cycle. Volume controlled ventilation may additionally have a set maximum flow rate that the apparatus is unable to increase the flow of gases above. A volume-controlled ventilation system may preferably comprise a proximal pressure and/or a flow sensor at the patient end of the system as described in more detail below. The delivered volume may be determined as described elsewhere in the system.

Resuscitation therapy is a form of respiratory therapy in which a patient is delivered a simulated breath for resuscitation purposes. It may be implemented by adjusting the set pressure of a respiratory therapy apparatus between a first pressure i.e. a set inspiratory pressure and second pressure i.e. a set expiratory pressure. For instance, the first pressure could be a positive inspiratory pressure (PIP) and the second pressure could be a positive end expiratory pressure (PEEP). The pressure adjustment between the first and second pressures could thereby be configured to simulate a breath. This may be achieved by a trigger of the respiratory therapy system that comprises for example a button, or a valve and/or an aperture. The trigger may be located for example on the patient interface, or on the expiratory conduit. A user or clinician may interact with the trigger to adjust the pressure between the first and second pressures and thus simulate a breath. For example, the system (and optionally the apparatus) may comprise a sensor as part of the trigger configured to provide a signal to the apparatus. The trigger may further be configured to transition the pressure between a first and a second pressure to simulate a breath, in response to the signal being detected. In some configurations the trigger may be located on a component of the system for example a connector. In some configurations, the trigger may be a mechanical trigger for example a valve which has a positive end expiratory pressure (PEEP) outlet which a user may occlude to adjust the pressure to the second pressure, and unocclude (such that gases flow through the PEEP outlet) to adjust the pressure to the first pressure.

An example of a component of the system as a connector which has a PEEP outlet that can be occluded is described in PCT application no. PCT/NZ2003/000014, PCT publication no. WO2003066146 which is hereby incorporated by reference in its entirety.

An example of a component of the system as a connector which has a trigger as a button which when interacted with produces a signal that causes the system to change between PEEP and PIP is described in PCT application no. PCT/NZ2020/050100, PCT publication no. WO2021049953, and PCT application no. PCT/IB2023/051252, PCT publication no. WO2023152712 which are hereby incorporated by reference in their entirety.

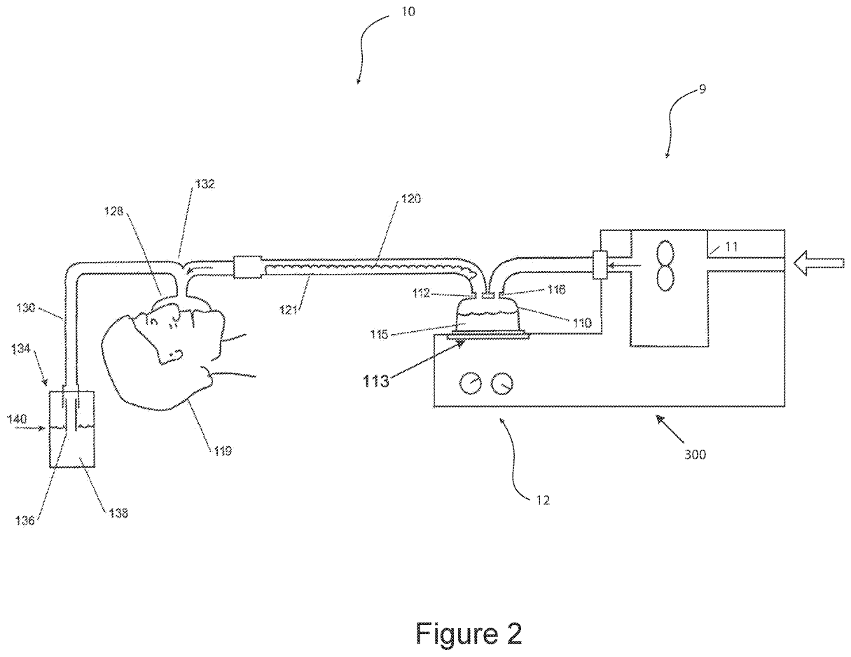

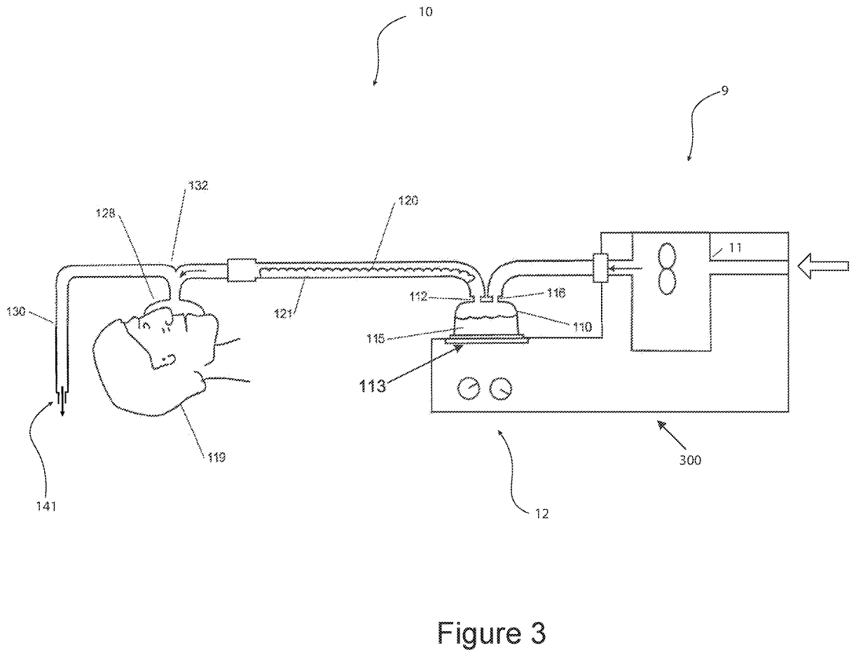

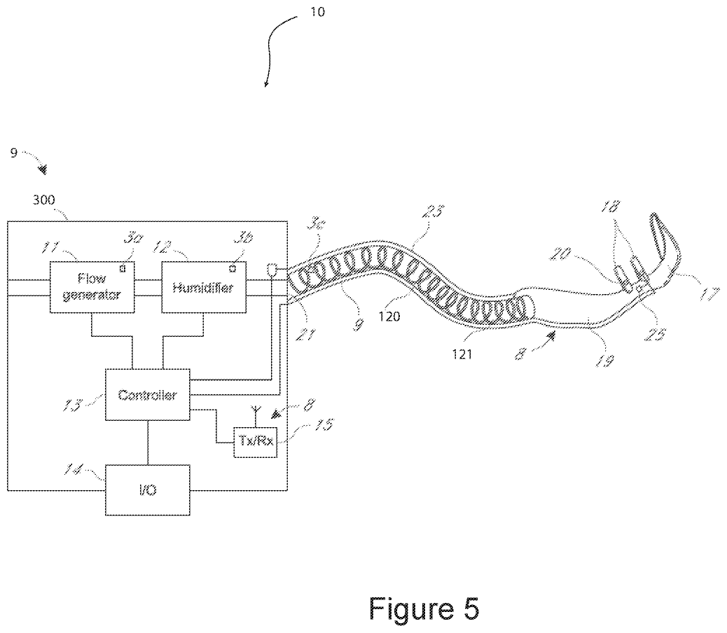

As described above, the breathing assistance apparatus may be able to provide various forms of therapy, such as a nasal high flow therapy (as shown in FIG. 5), a bubble CPAP therapy (as shown in FIG. 2), a variable flow CPAP therapy (as shown in FIGS. 3 and 4), a asynchronous and/or synchronous Nasal Intermittent Positive Pressure Ventilation therapy (as shown in FIGS. 3 and 4), a NIV-NAVA therapy (as shown in FIGS. 3 and 4), a HFOV therapy (as shown in FIGS. 3 and 4), a volume-limited pressure control therapy (as shown in FIGS. 3 and 4), a volume control ventilation therapy (as shown in FIGS. 3 and 4), and/or a resuscitation therapy (as shown in FIGS. 3 and 4), thereby making for an easier transition between different types of respiratory support as the patient's condition changes, and may also reduce the number of consumable components required, for example a common heated breathing tube may be used across multiple therapies, requiring only the patient interface to be changed (when required for example when transitioning from a mode with a sealed interface to a mode with an unsealed interface).

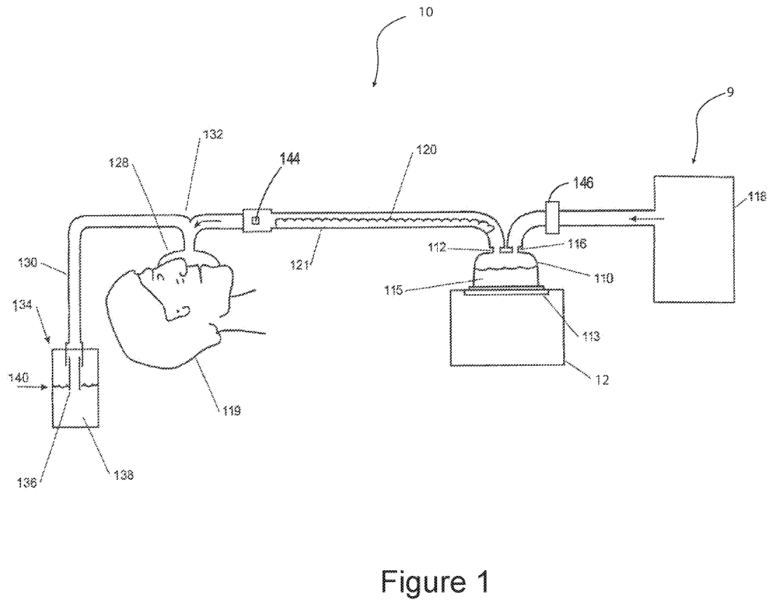

FIGS. 1 to 5 show the apparatus configured to provide various therapy modes as described above.

As shown in FIG. 1, a system for providing bubble CPAP therapy can provide to a patient 119 humidified and pressurized gas through a patient interface, such as a mask 128 in FIG. 1 connected to an inspiratory conduit 121. The inspiratory conduit 121 is connected to the outlet 112 of a humidifier 12 (i.e. the outlet of the humidification chamber 110), which contains a volume of water 115. As the volume of water 115 within the humidification chamber 110 is heated by a heater plate 113 in the humidifier 114, water vapor begins to fill the volume of the chamber 110 above the water's surface. The water vapor can heat and humidify a flow of gas (for example, air) provided from a wall source 118 (see FIG. 1) into the chamber 110 through an inlet 116 of the chamber 110. The heated and humidified gas is passed out of an outlet 112 of the humidification chamber 110 into the inspiratory conduit 121.

The humidified gas can pass through the inspiratory conduit 121 to a patient interface, such as the mask 128, attached and/or sealed around the patient's 119 mouth, nose, and/or nares. The inspiratory conduit 121 provides the patient 119 with a flow of gas that may by ambient air, oxygen, a mixture of the two, or a mixture of ambient air and other auxiliary gas(es). The gas may include medicaments, which may be added through nebulization. The flow of gas through the inspiratory conduit 121 can be delivered at a substantially constant flow rate in a bubble CPAP. As shown in FIG. 1, a setup has a flow of gas supplied by the wall source 118. The wall source 118 can deliver the gas at the target flow rate so as to maintain the flow rate of gas delivered to the patient.

As shown in FIGS. 1 to 5, the inspiratory conduit 121 may contain a heater, such as heater wires 120, which heat the walls of the conduit to promote a substantially constant humidity profile along the inspiratory conduit 121 and therefore reduce condensation of the humidified gas within the inspiratory conduit 121. The apparatus can supply power to heat the inspiratory conduit 121 and the heater plate 113, such as through input from one or more sensors in the system, as will be described in further detail below.

As shown in FIG. 1, excess gas can flow through the expiratory conduit 130 to a pressure regulator 134, which is a bubbler in the illustrated example. In a bubble CPAP system, the expiratory conduit 130 can terminate in an open terminal end 136. This terminal end 136 can be submerged in a volume of water 138 inside the bubbler 134. As described in more detail below the bubbler 134 comprises a chamber with a column of water and the expiratory conduit 130 being submerged into the column of water. The pressure provided to the user being defined or being set by the depth the submersion of the expiratory conduit 130 within the column of water.

The bubbler can regulate pressure by the terminal end 136 of the expiratory conduit 130 submerged at a desired depth under the water level 140 within the volume of water 138. The terminal end 136 can also optionally be located on a short conduit that can be integrated into the end of the expiratory conduit 130. The bubbler can act as a pressure regulator by venting out gas whenever the pressure exceeds the desired level so as to maintain the average or mean pressure at the target level. The bubble CPAP system can also include a pressure relief valve 146 for venting excess gas when the pressure exceeds the desired level. The bubbler can also provide oscillations in the pressure, which may have clinical benefits. Bubble CPAP therapy may lower the incidence of acute lung injury and bronchopulmonary dysplasia, compared with intubation and/or mechanical ventilation.

### Overview of Example Flow Therapy Apparatus

FIGS. 2 to 5 illustrate examples of a breathing assistance apparatus 9 with a flow generator 11 (also referred to as a blower but can include other types of flow generator disclosed herein) and humidifier 12. By integrating the humidifier, into the apparatus, fewer separate components are needed in the system and reduces the number of interconnections between devices, which simplifies its setup, and simplifies usability for a clinician. Further the system occupies less space because there are less separate components connected by tubes. Further still, a single controller can be provided which controls the therapy provided to the user (i.e. humidity, flow, pressure etc.). Having a single user interface to control the flow generator and humidifier may also simplify usability as well.

The system in FIG. 2 can also optionally include a supplementary gas source (such as an oxygen tank, an oxygen blender coupled to a flowmeter, and the like) for controlling oxygen concentration in the flow of gas delivered to the patient 119. The supplemental gas source can be connected to the apparatus 9 at a supplementary gas inlet, for example to the apparatus housing 214 and/or to the flow generator 11. The supplementary gas source can also be configured to provide other types of auxiliary gas, such as nitrogen. The supplementary gas source may be connected to an internal mixer (as described in more detail below) that blends ambient air and the supplementary gases to provide a gases flow to a patient. The concentration of the supplementary gases introduced into, or present in, the gases stream can be controlled.

The system 10 can include a temperature sensor, such as the temperature sensor 144 of FIG. 1, in the inspiratory conduit 121. The temperature sensor 144 can be coupled to and in electrical communication with a controller located in the apparatus housing 214.

The breathing assistance apparatus 9 as shown in FIGS. 2 to 5, with an integrated flow generator 11 and humidifier 12 and optionally an integrated supplementary gas mixer can occupy less space and reduce additional interconnecting tubes. Additionally, the integrated flow generator 11 and humidifier 12, and supplementary gases mixer can be controlled by a single controller, which allows for additional monitoring and control of various flow parameters, as will be described further.

In some embodiments a blower of the flow generator 11 is configured to receive the ambient gases and supplementary gases and mix these together. Having the blower mix the ambient and supplementary gases may further reduce the number of components required and simplifies use and setup. Further, having fewer components may decrease the resistance to flow of the circuit.

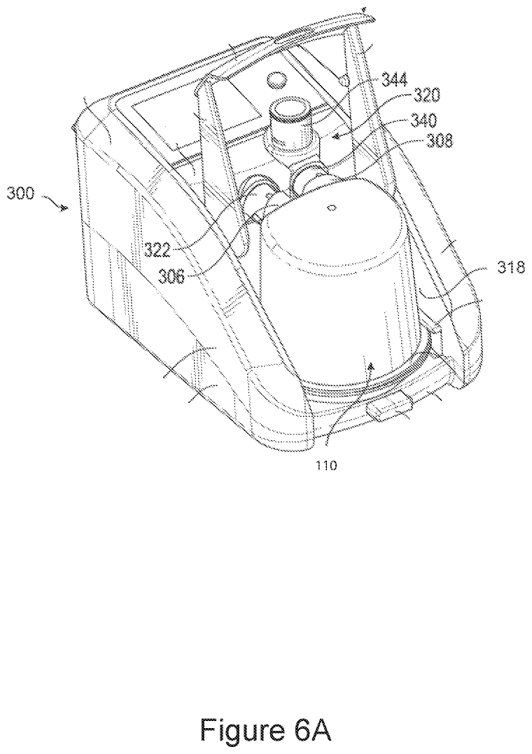

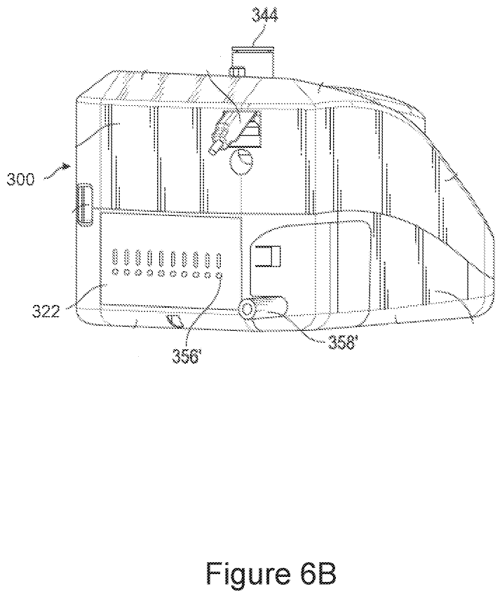

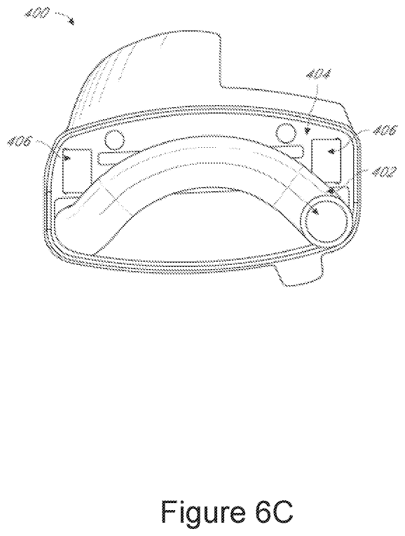

FIG. 2 illustrates an example breathing assistance apparatus 9 configured to provide bubble CPAP. Using a flow generator to generate the flow of gas can allow the breathing assistance apparatus to be used without a wall source to provide bubble CPAP, such as in circumstances where a wall source is not available. Further, using a flow generator e.g. a blower in a breathing assistance apparatus allows the breathing assistance apparatus to draw in ambient air and provide ambient air as a flow of gases for bubble CPAP. This allows the breathing assistance apparatus to be simpler and cheaper to use as there is no requirement for a gas store or a gas source e.g. a wall source. Further the breathing assistance apparatus with a flow generator e.g. a blower is advantageous because there is no risk of running out of gases, since ambient air is provided to the patient. This ensures there is no disruption in therapy due to a gas source being empty, since ambient air is abundant. The apparatus may also provide supplementary gases as part of the gases provide to the patient for example by integrating an oxygen inlet port 358′ shown in FIG. 6C. The

The respiratory system in FIG. 2 can differ from the conventional bubble CPAP setup in FIG. 1 at least by having the flow of gas provided by the flow generator 11 integrated within the apparatus 9 (for example within the apparatus housing 214.)

FIG. 3 shows an example of example breathing assistance apparatus 9 configured to provide variable flow CPAP therapy, and/or asynchronous and/or synchronous Nasal Intermittent Positive Pressure Ventilation therapy, and/or Non-Invasive Ventilation Neurally Adjusted Ventilatory Assist (NIV-NAVA) therapy, and/or High Frequency Oscillatory Ventilation (HFOV) therapy, and/or volume-limited pressure control therapy, and/or volume control ventilation therapy, and/or resuscitation therapy. The system 10 comprises a pressure regulator as an expiratory orifice 141. In FIG. 3 the expiratory conduit 130 is connected to the expiratory orifice 141.

The expiratory orifice 141 being at connected to the expiratory conduit 130 allows for any exhaled gases to be exhausted away from the patient. This means that in cases where the patient is neonate or infant located in an incubator exhaled gases can be exhausted external to the incubator.

FIG. 4 shows an example of example breathing assistance apparatus 9 configured to provide variable flow CPAP therapy, and/or asynchronous and/or synchronous Nasal Intermittent Positive Pressure Ventilation therapy, and/or NIV-NAVA therapy, and/or HFOV therapy, and/or volume-limited pressure control therapy, and/or volume control ventilation therapy, and/or resuscitation therapy. In FIG. 4 the expiratory gas flow is shown as venting through a vent in the mask 128. However it will be appreciated that the expiratory gases may travel through a connected expiratory conduit 130 (as for example shown in FIGS. 1 to 3) to a valve or other venting means.

It will be appreciated that variable flow CPAP, and/or asynchronous and/or synchronous Nasal Intermittent Positive Pressure Ventilation therapy, and/or NIV-NAVA therapy, and/or HFOV therapy, and/or volume-limited pressure control therapy, and/or volume control ventilation therapy, and/or resuscitation therapy, may be provided with the example system as shown in FIGS. 3 and 4. This means that variable flow CPAP therapy and synchronous Nasal Intermittent Positive Pressure Ventilation therapy, and NIV-NAVA therapy, and HFOV therapy, and volume-limited pressure control therapy, and volume control ventilation therapy, and resuscitation therapy can be provided using the same conduit and interface set up. It will be appreciated that in some configurations where the system shown in FIGS. 3 and 4 is configured to provide resuscitation therapy, the system may further comprise a trigger. The trigger may for example comprise a button, or a valve and/or an aperture. For instance, the trigger (for example the button, or the valve and/or the aperture) may be located on or proximate the mask 128, or the expiratory conduit 130.

It will further be appreciated that in some configurations where the system shown in FIGS. 3 and 4 is configured to provide NIV-NAVA therapy, the system may further comprise at least one physiological sensor (for example as a patient sensor). The at least one physiological sensor may for instance be located on the patient interface 128, or the expiratory conduit 130, or directly on the patient (for example as a diaphragmatic sensor as described in more detail elsewhere in the specification).

In FIGS. 1 to 4, the patient interface (mask 128) is a sealing interface. This means that it may be possible to switch between different therapies without changing the interface. In some examples, the patient interface may be a nasal interface. The patient interface may be for example a sealing interface like a face mask, an oro-nasal mask, a nasal mask, a nasal pillow mask, or a nasal cannula.

In some examples the interface is a nasal cannula comprising a pair of sealing nasal prongs configured to provide the gases flow to the patient's nares.

FIG. 5 is a schematic view of an apparatus and system similar to that of FIGS. 1-4. The apparatus and/or system 10 shown in FIG. 5 may have features of the system of FIGS. 1-4, and the apparatus and/or system 10 shown in FIGS. 1-4 may have features of the system of FIG. 5.

Referring to FIGS. 1-5, the interface may correspond the type of therapy provided.

The respiratory system in FIG. 5 shows the apparatus configured as a high flow system 10. A schematic representation of a high flow system 10 is provided in FIG. 5. The respiratory system 10 can include an apparatus housing 214. The apparatus housing 214 can contain a flow generator 11 that can be in the form of a motor/impeller arrangement (such as a blower), a humidifier 12, a controller 13, and a user interface 14. The user interface 14 can include a display and input device(s) such as button(s), a touch screen, a combination of a touch screen and button(s), or the like. The controller 13 can include one or more hardware and/or software processors and can be configured or programmed to control the components of the apparatus, including but not limited to operating the flow generator 11 to create a flow of gas for delivery to a patient, operating the humidifier 12 humidify and/or heat the gas flow, receiving user input from the user interface 14 for reconfiguration and/or user-defined operation of the respiratory system 10, and outputting information (for example on the display) to the user. The user can be a patient, healthcare professional, or others.

With continued reference to FIG. 5, a inspiratory conduit 121 can be coupled to a gases flow outlet 21 in the apparatus housing 300 of the respiratory system 10, and be coupled to a patient interface 17. The patient interface can be a non-sealing interface like a nasal cannula with a manifold 19 and nasal prongs 18 for providing a high flow therapy. The nasal cannula does not completely seal with the nostrils of the user such that exhaled gases leak out from around the nasal prongs when the user exhales. The inspiratory conduit 121 can also be coupled to a sealing interface like a face mask, an oro-nasal mask, a nasal mask, a nasal pillow mask, or a nasal cannula for providing bubble CPAP. The patient interface can also optionally include an endotracheal tube, a tracheostomy interface, or others.

The flow of gas can be generated by the flow generator 11, and may be humidified, before being delivered to the patient via the inspiratory conduit 121 through the patient interface 17. The controller 13 can control the flow generator 11 to generate a gas flow of a desired flow rate, and/or one or more valves to control mixing of air and oxygen or other breathable gas. The controller 13 can control a heating element in the humidifier 12, if present, to heat the gases to a desired temperature that achieves a desired level of temperature and/or humidity for delivery to the patient. The inspiratory conduit 121 can have a heating element 120, such as a heater wire, to heat gases flow passing through to the patient. The heating element 120 can also be under the control of the controller 13. The heating element 120 heats gases to reduce and/or prevent condensation within the inspiratory conduit 121.

As described above the system may comprise a heater in inspiratory conduit 121.

The inspiratory conduit 121 and/or expiratory conduit 130 may comprise a heater.

The heater may be a heater wire as for example shown in FIGS. 1 to 5.

The heater wire may be located: in a passageway of the inspiratory conduit and/or expiratory conduit, attached to a wall of the inspiratory conduit and/or expiratory conduit, embedded in a wall of the inspiratory conduit and/or expiratory conduit.

The system 10 can use ultrasonic transducer(s), flow sensor(s) such as a thermistor flow sensor, pressure sensor(s), temperature sensor(s), humidity sensor(s), or other sensors, in communication with the controller 13, to monitor characteristics of the gas flow and/or operate the system 10 in a manner that provides suitable therapy. The gas flow characteristics can include gases concentration, flow rate, pressure, temperature, humidity, or others. The sensors 3a, 3b, 3c, 20, 25, such as pressure, temperature, humidity, and/or flow sensors, can be placed in various locations in the apparatus housing 300, the patient conduit 16, and/or the patient interface 17. The controller 13 can receive output from the sensors to assist it in operating the respiratory system 10 in a manner that provides suitable therapy, such as to determine a suitable target temperature, flow rate, and/or pressure of the gases flow. Providing suitable therapy can include meeting a patient's inspiratory demand. The suitable therapy flow rates, such as a high flow therapy flow rate, and/or a flow rate meeting or exceeding the patient's inspiratory demand, are explained below.

The system 10 can include a wireless data transmitter and/or receiver, or a transceiver 15 to enable the controller 13 to receive data signals 8 in a wireless manner from the operation sensors and/or to control the various components of the system 10. Additionally, or alternatively, the data transmitter and/or receiver 15 can deliver data to a remote server or enable remote control of the system 10. In one example the remote server can record patient usage data e.g. usage of the bubble CPAP system or usage of the high flow system. Usage can be usage time and/or also include flow rate and humidity level (e.g. dew point). The system 10 can also include a wired connection, for example, using cables or wires, to enable the controller 13 to receive data signals 8 from the operation sensors and/or to control the various components of the system 10.

In some examples, the system may comprise a pressure sensor configured to measure a pressure at or near the patient. The pressure sensor may be located in a conduit connected to the patient interface (for example at or near the patient interface). The pressure sensor may be located in the patient interface 128. The pressure sensor may be located in the apparatus 300, and a pressure conduit may be connected between the apparatus and the inspiratory conduit 121 or patient interface 128. The pressure conduit may provide a pathway for changes in pressure in the inspiratory conduit 121 or patient interface 128 to be communicated to the pressures sensor.

Patient sensors (for example as described elsewhere in the specification) may be connected to the apparatus for example via a wireless or wired connection.

It will be appreciated that the features of the apparatuses 9 as shown in FIG. 5 may be applicable to the apparatus shown in FIGS. 1 to 4. Similarly, features of the apparatus 9 of FIG. 5 may be applicable to the apparatuses 9 of FIGS. 1 to 4.

For example:

The controller 13 as shown in FIG. 5, may also be provided as part of the system (for example as part of the apparatus) in the systems of FIGS. 1 to 4. The controller may be configured to control components of the system 10.

The user interface 14 as shown in FIG. 5, may also be provided as part of the system (for example as part of the apparatus) in the systems of FIGS. 1 to 4.

The data transmitter and/or receiver 15 as shown in FIG. 5, may also be provided as part of the system (for example as part of the apparatus) in the systems of FIGS. 1 to 4.

Other sensors as described elsewhere in the specification may additionally or alternatively be provided in the systems as shown in FIGS. 1 to 5.

The system 10 can be powered from mains voltage.

In some embodiments, the system can include an auxiliary power source (for example a battery).

In some embodiments, the system can include a battery. The battery may provide the main source of power for the system, or may serve as an auxiliary source of power when the main source of power is unavailable. This is advantageous because therapy can be continued to be delivered i.e. gases can be continue to be delivered to a patient even if there is a shortage or outage in mains power. This is advantageous because therapy can be maintained for a period of time for neonatal or infants thereby reducing the chances or physiological deterioration or harm occurring to these patient's due to loss of therapy.

The battery can increase portability of the system to allow for the system to be used in situations where a mains voltage power source is unavailable.

Having a battery power source, allows for the apparatus to be portable. This may be useful in a hospital setting where infant/neonate patients may need to be moved.

The battery may also allow for the therapies described herein to be provided continuously while the patient is moved. For example a therapy type may be able to be changed as described below, while the patient is moved, while the patient is continuously provided with respiratory therapy.

The housing 214, 300 may comprise:
a blower of the flow generator,
a humidifier,
the controller of the apparatus,
an oxygen sensor,
a gases mixer,
a battery,
a supplemental gases inlet,
an ambient air outlet,
any combination of the above.

FIGS. 6A and 6B show an example breathing assistance apparatus 9 of the respiratory system 10. The apparatus can include a housing 300, which encloses a flow generator. The flow generator may include a motor and/or sensor module. The motor and/or sensor module may be non-removable from the main housing 300. The motor and/or sensor module can also optionally be removable from the main housing 300. The housing 300 can include a humidifier or humidification chamber bay 318 for receipt of a removable humidification chamber 110. The removable humidification chamber 110 contains a suitable liquid such as water for heating and humidifying gases delivered to a patient. The humidification chamber 110 can be fluidly coupled to the main housing 300 in a linear slide-on motion into the chamber bay 318. A gas outlet port 322 can establish a fluid communication between the motor and/or sensor module and an inlet 306 of the chamber 110.

Heated and humidified gas can exit an outlet 308 of the chamber 110 into a humidified gas return 340, which can include a removable L-shaped elbow. The removable elbow can further include a patient outlet port 344 for coupling to the inspiratory conduit, such as the inspiratory conduit 16 of FIG. 5 to deliver gases to the patient interface 17. The gas outlet port 322, humidified gas return 340, and patient outlet port 344 each can have seals such as O-ring seals or T-seals to provide a sealed gases passageway between the apparatus housing 300, the humidification chamber 110, and the inspiratory conduit. A floor portion of the humidification chamber bay 318 in the housing 300 can include a heater arrangement such as a heater plate or other suitable heating element(s) for heating the water in the humidification chamber 110 for use during a humidification process. The elbow may comprise one or more integrated sensors. For example the elbow may comprise a pair of embedded temperature sensors.

As shown in FIG. 6B, the apparatus can include an arrangement to enable the flow generator to deliver air, oxygen (or alternative auxiliary gas), or a suitable mixture thereof to the humidification chamber 110 and thereby to the patient. This arrangement can include an air inlet 356′ in a rear wall of the housing 300. The apparatus can include a separate oxygen inlet port 358′. In the illustrated configuration, the oxygen inlet port 358′ can be positioned adjacent one side of the housing 300 at a rear end thereof. The oxygen port 358′ can be connected to an oxygen source such as a tank, or an oxygen blender. The oxygen inlet port 358′ can be in fluid communication with a valve. The valve can suitably be a solenoid valve that enables the control of the amount of oxygen that is added to the gas flow that is delivered to the humidification chamber 110.

The housing 300 can include suitable electronics boards, such as sensing circuit boards. The electronics boards can contain, or can be in electrical communication with, suitable electrical or electronics components, such as but not limited to microprocessors, capacitors, resistors, diodes, operational amplifiers, comparators, and switches. One or more sensors can be used with the electronic boards. Components of the electronics boards (such as but not limited to one or more microprocessors) can act as the controller 13 of the apparatus. One or both of the electronics boards can be in electrical communication with the electrical components of the system 10, including but not limited to the display unit and user interface 14, motor, valve, and the heater plate to operate the motor to provide the desired flow rate of gases, humidify and heat the gases flow to an appropriate level, and supply appropriate quantities of oxygen (or quantities of an alternative auxiliary gas) to the gases flow.

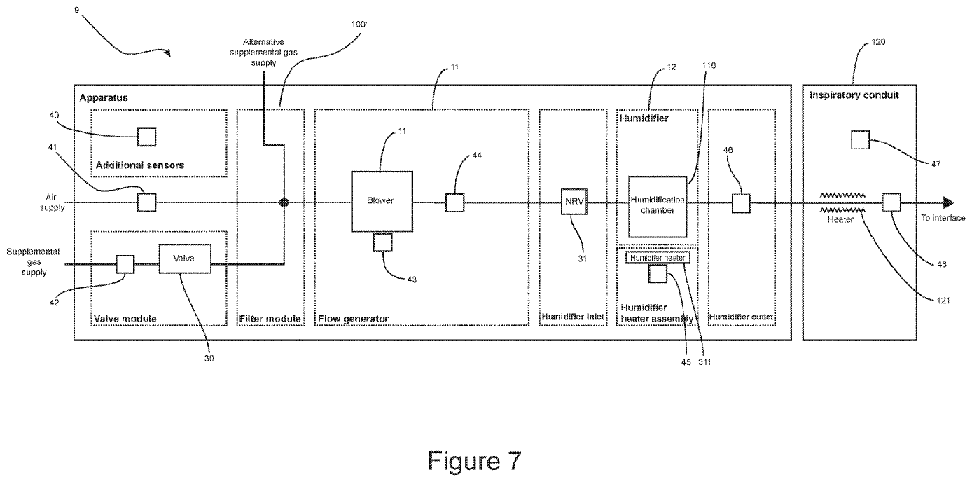

As mentioned elsewhere in the specification and for example with respect to FIG. 7, operation sensors, such as flow, temperature, humidity, and/or pressure sensors can be placed in various locations in the breathing assistance apparatus, the patient conduit 16, and/or cannula 17. The electronics boards can be in electrical communication with those sensors. Output from the sensors can be received by the controller 13, to assist the controller 13 to operate the respiratory system 10 in a manner that provides optimal therapy, including meeting inspiratory demand. One or more sensors (for example, Hall-effect sensors) may be used to measure a motor speed of the motor of the flow generator. The motor may include a brushless DC motor, from which motor speed can be measured without the use of separate sensors. For example, during operation of a brushless DC motor, back-EMF can be measured from the non-energized windings of the motor, from which a motor position can be determined, which can in turn be used to calculate a motor speed. In addition, a motor driver may be used to measure motor current, which can be used with the measured motor speed to calculate a motor torque. The motor may also include a low inertia motor.

Room air can enter the flow generator through the inlet port, such as the air inlet port 356′ in FIG. 6B. The flow generator can operate at a motor speed of greater than 1,000 RPM and less than 30,000 RPM, greater than 2,000 RPM and less than 21,000 RPM, greater than 4,000 RPM and less than 15000 RPM, or between any of the foregoing values. Operation of the flow generator can mix the gases entering the flow generator, such as the motor and/or sensor chamber through the inlet port. Using the flow generator as the mixer can reduce the pressure drop that would otherwise occur in a system with a separate mixer, such as a static mixer comprising baffles, because mixing requires energy.

As shown in FIG. 6C, the mixed air can exit the flow generator and enter a flow path 402 in a sensor chamber 400, which can be located in the motor and/or sensor module. A sensing circuit board 404 with sensors, such as ultrasonic sensors 406 and/or heated thermistor flow sensors, can be positioned in the sensor chamber 400 such that the sensing circuit board is at least partially immersed in the gas flow. At least some of the sensors on the sensing circuit board can be positioned within the gas flow to measure gas properties within the flow. After passing through the flow path 402 in the sensor chamber 400, the gas can exit to the humidification chamber 110.

Positioning sensors downstream of the flow generator (as described in more detail below) can increase accuracy of measurements, such as the measurement of gases fraction concentration, including oxygen concentration, over systems that position the sensors upstream of the flow generator and/or the mixer. Such a positioning can give a repeatable flow profile. Further, positioning the sensors downstream of the combined flow generator and mixer avoids the effect of the pressure drop that would otherwise occur when sensing occurs prior to the flow generator and a separate mixer. Also, immersing at least part of the sensing circuit board and sensors in the flow path can increase the accuracy of measurements because the sensors being immersed in the flow are more likely to be subject to the same conditions, such as temperature and pressure, as the gas flow and therefore provide a better representation of the gas flow characteristics.

As shown in FIG. 6C, the flow path 402 can have a curved shape. The flow path 402 can be configured to have a curved shape with no sharp turns. The flow path 402 can have curved ends with a straighter section between the curved ends. A curved flow path shape can reduce pressure drop in a gas flow without reducing the sensitivity of flow measurements by partially coinciding a measuring region with the flow path to form a measurement portion of the flow path.

The sensing circuit board 404 can include sensors such as acoustic transmitters and/or receivers, humidity sensor, temperature sensor, thermistor, and the like. A gas flow rate may be measured using at least two different types of sensors. The first type of sensor can include a thermistor, which can determine a flow rate by monitoring heat transfer between the gases flow and the thermistor. The thermistor flow sensor can run the thermistor at a constant target temperature within the flow when the gas flows around and past the thermistor. The sensor can measure an amount of power required to maintain the thermistor at the target temperature. The target temperature can be configured to be higher than a temperature of the gas flow, such that more power is required to maintain the thermistor at the target temperature at a higher flow rate.

The thermistor flow rate sensor can also maintain a plurality of (for example, two, three, or more) constant temperatures on a thermistor to avoid the difference between the target temperature and the gas flow temperature from being too small or too large. The plurality of different target temperatures can allow the thermistor flow rate sensor to be accurate across a large temperature range of the gas. For example, the thermistor circuit can be configured to be able to switch between two different target temperatures, such that the temperature of the gas flow can always fall within a certain range relative to one of the two target temperatures (for example, not too close and not too far). The thermistor circuit can be configured to operate at a first target temperature of about 50° C. to about 70° C., or about 66° C. The first target temperature can be associated with a desirable flow temperature range of between about 0° C. to about 60° C., or about 0° C. and about 40° C. The thermistor circuit can be configured to operate at a second target temperature of about 90° C. to about 110° C., or about 100° C. The second target temperature can be associated with a desirable flow temperature range of between about 20° C. to about 100° C., or about 30° C. and about 70° C.

The controller can be configured to adjust the thermistor circuit to change between at least the first and second target temperature modes by connecting or bypassing a resistor within the thermistor circuit. The thermistor circuit can be arranged as a Wheatstone bridge configuration comprising a first voltage divider arm and a second voltage divider arm. The thermistor can be located on one of the voltage divider arms. More details of a thermistor flow rate sensor are described in International Patent No. WO2018052320A2, the entirety of which is incorporated by reference herein.

The second type of sensor can include an acoustic (such as ultrasonic) sensor assembly. Acoustic sensors including acoustic transmitters and/or receivers can be used to measure a time of flight of acoustic signals to determine gas velocity and/or composition, which can be used in flow therapy apparatuses. In one ultrasonic sensing (including ultrasonic transmitters and/or receivers) topology, a driver causes a first sensor, such as an ultrasonic transducer, to produce an ultrasonic pulse in a first direction. A second sensor, such as a second ultrasonic transducer, receives this pulse and provides a measurement of the time of flight of the pulse between the first and second ultrasonic transducers. Using this time of flight measurement, the speed of sound of the gas flow between the ultrasonic transducers can be calculated by a processor or controller of the breathing assistance apparatus. The second sensor can also transmit and the first sensor can receive a pulse in a second direction opposite the first direction to provide a second measurement of the time of flight, allowing characteristics of the gas flow, such as a flow rate or velocity, to be determined. In another acoustic sensing topology, acoustic pulses transmitted by an acoustic transmitter, such as an ultrasonic transducer, can be received by acoustic receivers, such as microphones. More details of an acoustic flow rate sensor are described in International Patent No. WO2017095241A3, which is incorporated by reference herein in its entirety. The acoustic pulses can be transmitted along the flow path of the gases, thereby allowing the acoustic sensors to be used to measure a flow rate or velocity of the gases.

Readings from both the first and second types of sensors can be combined to determine a more accurate flow measurement. For example, a previously determined flow rate and one or more outputs from one of the types of sensor can be used to determine a predicted current flow rate. The predicted current flow rate can then be updated using one or more outputs from the other one of the first and second types of sensor, in order to calculate a final flow rate.

### Controlling FdO₂

As described above, the flow generator can be used as an oxygen and/or other breathable gas mixer. The flow generator that draws in ambient air (for example from an ambient air inlet as described in more detail below) can mix the air with oxygen from an oxygen source. This oxygen source can be from a high pressure source (for example the supplemental gas inlet as described in more detail below) or a low pressure source (for example the alternative gas inlet as described in more detail below).

When receiving oxygen from low pressure source, which may include an oxygen canister or tank, an oxygen wall source, or an oxygen concentrator, the breathing assistance apparatus can receive a constant flow rate of oxygen. This oxygen can then be mixed with ambient air. The fraction of oxygen in the gas delivered to the patient (FdO2) can be dependent on the set flow rate of oxygen from the low pressure source, and the total flow rate that the apparatus generates. The apparatus can measure FdO2 and display it on the display.

When receiving oxygen from a high pressure source, which may include an oxygen canister or tank, an oxygen wall source, or an oxygen concentrator, the device can control the flow rate of oxygen by controlling the valve to the oxygen inlet port described herein. The FdO2 can be dependent on the flow rate of oxygen through the valve (which can be further dependent on the state of the valve opening), and on the total flow rate that the apparatus generates. A user, such as the clinician, can set a target FdO2 on a user interface of the display, with the apparatus then controlling the valve opening based on the target FdO2 and measured FdO2 in order to achieve the desired fraction of oxygen.

It will be appreciated that in instances where the system is configured to measure and control the FdO2 of the delivered gas, the system may additionally or alternatively be configured to control the fraction of oxygen in the gas inspired by the patient (FiO2).

Oxygen concentration can be measured by a variety of sensors, such as using the ultrasonic sensors described above. More details of example methods of measuring the oxygen concentration are described in International Patent No. WO2013151447A1, the entirety of which is incorporated herein by reference.

A patient parameter indicative of oxygen saturation (SpO2) may be measured by and received from at least one sensor. The FdO2 of the gases delivered to the patient may be based at least in part on the patient parameter. A gases composition sensor may be configured to measure at least the FdO2 of the delivered gases. The gases composition sensor could be, for example, an ultrasonic sensor.

In some examples, the system may comprise a pulse oximeter. The apparatus may be configured to connect to the pulse oximeter. The controller may be configured to calculate a patient's oxygen saturation based on at least an output of the pulse oximeter.

The apparatus may control an oxygen concentration of the gases (for example by controlling the valve) to control a patient's oxygen saturation to a target patient's oxygen saturation. The controller 13 may use the pulse oximeter in feedback to control the patient's oxygen saturation.

In some configurations the apparatus may be configured to allow a user to control the apparatus to deliver 100% FdO2 for a predetermined period. For instance, the apparatus may comprise a button (or other that a clinician or patient may press to cause the apparatus to deliver 100% FdO2 for the given predetermined period.

### Controlling Flow Rate and Pressure

In some configurations, blower and/or motor parameters may be controlled by flow generator to maintain the flow rate at a desired level (i.e. a set flow rate).

In some configurations, blower and/or motor parameters may be controlled by flow generator to maintain the pressure of the flow of gases at a desired level (i.e. a set pressure).

The set pressure may be a pressure at or near the patient and/or at a patient end of an inspiratory conduit.

The pressure at or near the patient and/or at a patient end of an inspiratory conduit may be measured directly, or for example estimated based on other sensor inputs (such as flow and/or pressure and/or motor speed measured downstream of the flow generator). In some examples, the pressure at or near the patient and/or at a patient end of an inspiratory conduit may be based on measurement of pressure at an output of the flow generator, and an estimated pressure drop from the measured pressured to the patient and/or at a patient end of an inspiratory conduit. The estimated pressure drop may be predefined and stored in memory.

For example, the flow generator (or for example the controller) may control one or more of the motor speed, motor current, and/or motor voltage to a target motor speed, a target motor current, and/or a target motor voltage.

In some configurations, the breathing assistance apparatus examples disclosed herein can determine the flow rate of gas in the gases pathway, and control the motor speed of the flow generator based at least in part on the flow rate measurement in order to maintain the flow rate at a desired level.

The flow generator may for example control the motor speed of the motor to a target motor speed. The motor speed can correspond to a desired flow rate i.e. target flow rate. In one example motor speed is a control parameter because the controller may be able to achieve faster response since feedback from motor speed can be quickly read by the controller as compared to feedback from a sensor e.g. flow sensor downstream of the blower. Alternatively, the controller may use the flow reading from the flow sensor to control the motor.

The controller controls the blower to a target motor speed or a target flow or both. The controller preferably provides control signals to control i.e. vary the current or voltage or power provided to the motor of the blower in order achieve the target motor speed or target flow rate.

As a further alternative the controller may use a combination of motor speed and flow rate to control the motor. In this example, the controller may use feedback from the motor speed reading and the flow reading from a flow sensor to control the motor to achieve a target motor speed and/or a target flow rate.

Measuring the flow rate can be done by using one or more flow rate sensors. As described above, examples of sensors that can measure a flow rate of gas can include ultrasonic sensors and heated thermistors. Ultrasonic sensors can provide a faster signal, but are generally less accurate than heated thermistors. Heated thermistors can provide a more accurate signal, but may not respond to small quick changes in the flow. In some examples, the output of multiple sensors (for example the ultrasonic sensor and heater thermistor) are used in the determined of the measured flow rate. In systems described herein, the flow signal of the flow sensor may need to be filtered before being used to control the flow generator. This is because a patient receiving therapy may cause fluctuations in the flow by coughing, talking, changing the cannula's positioning, etc. In this case, it can be desirable that the apparatus does not make sudden changes to the motor speed based on these events.

However, when delivering bubble CPAP, these patient-caused fluctuations in the flow are less common. Therefore, the control of the flow rate can be designed to be more responsive to account for small leaks in the system, partial blockages, and/or dynamic changes in the patient's respiratory demand. The flow rate control can be done by using a shorter filter than in the nasal high flow therapy on the flow rate measurement. Additionally, and/or alternatively, the controller can use the flow rate measured by the ultrasonic sensors to provide a much faster signal. Additionally, and/or alternatively, the controller can use a combination of flow rates measured by the ultrasonic sensors and the heated thermistor. The controller can use the ultrasonic sensors to detect higher frequency changes in the flow rate, and use the heated thermistor to compensate for the lower accuracy measurements made by the ultrasonic sensors, which may be less accurate than when measured by the heated thermistor. The controller can also use other types of sensor(s) for measuring flow rate and/or pressure. The pressure and/or flow rate sensor can include a single sensor.

Once the system controller receives flow signals from the one or more flow rate sensors disclosed herein, the controller can measure the flow rate from the flow signals. The system controller can determine a difference between a target flow rate and the measured flow rate. The differences can be input into a PID controller. The PID controller can output a command to change to the motor speed of the flow generator based on the inputs. In one example the PID controller may output a current or voltage or power to the motor in order to control the motor speed.

The control of the flow rate can also be based on pressure measurements. In a conventional bubble CPAP system, a pressure relief valve is placed between the flow source and the patient. The pressure relief valve is a passive valve that can open at a set pressure in order to vent off a portion of the gas flow, thereby limiting the pressure of the gas delivered to the patient.

By using a flow generator to provide the flow of gas, the pressure limit can be implemented on the motor speed of the flow generator via software. The high flow system described herein may not include an additional valve for venting of excess flow. The control of the motor speed can provide a more accurate control of the pressure than the venting of the gas through the pressure relief valve, as the motor speed of the flow generator can be directly controlled based on the measured pressure. The use of a respiratory system (i.e. a breathing assistance apparatus) with a flow generator, as described herein, does not require a pressure relief valve since the flow generator can be controlled to reduce the motor speed to generate less pressure if a pressure limit is reached. The system is simplified and requires less components since the system does not require a pressure relief valve and also does not require a gases source.

In modes where the apparatus controls to a set pressure (for example variable flow CPAP mode and/or synchronous or synchronous Nasal Intermittent Positive Pressure Ventilation), a particular control scheme may be utilized.

The control scheme may default to pressure control, however if leak cannot be compensated for in pressure control, then the apparatus may utilize flow control. This may be beneficial as it means the patient (typically an infant) will still receive some degree of therapy even where a leak is detected. Conventionally, in pressure-based systems the delivery of therapy is typically stopped when a leak is detected. However, where the patient is a neonate, it may be desirable to continue to deliver therapy even where there is a leak, which may arise for instance from a mask fit issue. Therefore, rather than cease to provide therapy entirely, the system may simply stop providing pressure therapy and switch to flow control.

In pressure control the apparatus provides the gases flow at a set pressure (i.e. in variable flow CPAP) or set pressures (for example inspiratory and expiratory pressure as in Nasal Intermittent Positive Pressure Ventilation).

As described above, the controller 13 may control the flow generator 11 to provide the flow of gases a set flow rate (i.e. flow control) or at a set pressure (i.e. pressure control).

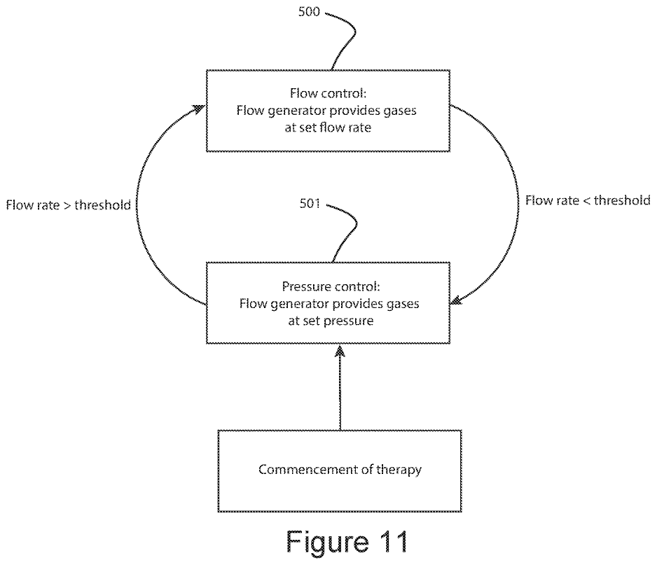

As for example as shown in FIG. 11, at commencement of therapy the apparatus is configured to attempt to control the flow generator according to pressure control. If during pressure control the determined flow rate exceeds the flow rate threshold (described in more detail below) the apparatus will instead control the flow generator according to flow control.

During therapy, the controller may control the flow generator to provide the flow of gases at a set flow rate (i.e. flow rate control 500) if a determined flow rate is greater than a flow rate threshold and control the flow generator to provide the flow of gases at a set pressure (i.e. pressure control 501) if the determined flow rate is lower than the flow rate threshold.

The set flow rate may be a function of the set pressure. In some examples the set flow rate is proportional to the square root of the set pressure (for example when the set pressure is the set pressure in a variable flow CPAP mode).

In some examples for example as set out below, the set flow rate of is a function of the set pressure and a conductance of the pressure regulator, and a conductance of the unintentional leak.

$$Set flow rate = ( {Conductance}_{Pressure regulator} + {Conductance}_{Unintentional leak} ) \sqrt{set pressure}$$

The conductance for the pressure regulator may define a relationship between an expected flow and the Square root for a set pressure.

The conductance for the unintentional leak may define a relationship between an expected leak flow and the square root for a set pressure.

The set flow rate may provide pressure support where leak cannot be compensated for in pressure control (for example the flow rate is too high for a given set pressure).

The user may set the set flow rate for example via a user interface.

The controller may be configured to raise an alarm when the controller controls the flow generator according to flow control. This may prompt a clinician to review why the apparatus is not controlling according to pressure control (as per normal operation).

The flow rate threshold is a maximum allowed leak flow rate. The maximum allowed leak flow rate may correspond to the sum of the flow caused by unintentional leak and the flow through the expiratory orifice. If the flow rate is larger than this then it is a sign that the flow generator cannot compensate for the leak.

The flow through the pressure regulator may be a function of a conductance of the pressure regulator and a determined pressure.

The flow from unintentional leak may be a function of a conductance of the unintentional leak and the determined pressure.

In some examples, the determined pressure is a pressure at or near the patient end of the or an inspiratory conduit (which may be measured directly, or estimated as described in more detail elsewhere in the specification).

The flow rate threshold may be calculated based on the equation below.

$$Flow rate threshold = ( {Conductance}_{Pressure regulator} + {Conductance}_{Unintentional leak} ) \sqrt{determined pressure}$$

The conductance for the pressure regulator may define a relationship between the expected flow and the square root of a determined pressure.

The conductance for the unintentional leak may define a relationship between an expected leak flow and the square root of determined pressure.

If the leak is too large in pressure control additional noise may be generated caused by the leaking flow. It may also be uncomfortable for a user.

If the flow rate is higher than anticipated for a given determined pressure, this is indicative that the leak is too great to be compensated for in pressure control. Continuing to try and provide pressure support in this case may be dangerous as for example if the leak is for example caused by the interface being detached, and the interface is reattached, then the increase in flow (to compensate for the leak that has been removed) may cause barotrauma or be uncomfortable.

The flow rate threshold may be dynamic. For example, as the flow rate threshold is dependent on the determined pressure, it may change across a breath (for example in the NIPPV modes where a higher inspiratory pressure is provided). In some examples, the controller recalculates the flow rate threshold periodically. In some examples, the controller changes the flow rate threshold value as part of the control scheme.

The flow rate threshold may be continuously compared to the determined flow rate.

The controller 13 may be configured to control the flow generator according to pressure control when the determined flow rate is lower than the flow rate threshold for a time period. In some examples, the time period is about 0.3 seconds to about 1 second or about. 0.3 seconds

The controller may be configured to control the flow generator according to flow control when the determined flow rate is greater than the flow rate threshold for a time period. In some examples, the time period is about 0.3 seconds to about 1 second or about 0.3 seconds

FIG. 7 shows a schematic view of the apparatus 9. FIG. 7 shows various locations of sensors 40, 41,42, 43, 44, 45, 46, 47 and 48 in the system which are described in more detail below.

It will be appreciated that any sensors may be provided as part of the apparatus, or as sensors external and connectable to the apparatus (for example as sensor probes). The inspiratory conduit and/or expiratory conduit may comprise a port, and wherein the sensor (for example the gases flow property sensor) is insertable into the port to be in contact with the gases in the gases flow path.

A gases flow path may be provided from one or more inlets through the filter module 1001 to the patient interface via the flow generator 11, humidifier 12 and inspiratory conduit 121.

The sensors may comprise one or more gases property sensors. The gases property sensors may be configured to measure a property of the gases flow in the gases flow pathway.

The gases flow pathway may be defined by a number of components in the system. For example the gases flow pathway may be defined by components which provide a pathway for gases downstream of the flow generator (for example as shown in FIG. 7).

The gases flow pathway may be for example defined by one or any combination of:
a portion of the flow generator downstream of the blower,
a humidifier,
an elbow comprising for example a patient outlet port 344,
an inspiratory conduit
a Wye piece
a patient interface
an expiratory conduit
a pressure regulator.

It will be appreciated that the above list is not exhaustive, and other components may form part of the gases flow pathway.

The gases property sensors may generate an output indicative of the property that they measure. The output may be provided to the controller. The controller may use the output to determine a property of the gases at the location of the sensor, or based on the output (and optionally other variables) determine a property of the gases at another location in the gases flow path.

The output of one or more gases property sensors may be filtered to remove unwanted noise.

The controller 13 may also the determined property of the gases to control the system.

Additionally, or alternatively to the specific examples below, the gases property sensors may be located for example
in a patient interface 128, 17,
in a pressure regulator 134, 141,

in the inspiratory conduit 121 and/or the expiratory conduit 130

The apparatus may comprise a number of gases property sensors, located at various locations in the system. For example, the sensors 44 may comprise a flow rate sensor and a pressure sensor.

The gases property sensors may for example include a temperature sensor, a pressure sensor and/or flow rate sensor.

In some examples, for example as shown with sensors 44, 46 and 48, the gases property sensors may be located downstream of the blower 11′.

As described above the apparatus 9 may receive air (for example ambient air via an air inlet port 27). The air inlet may comprise at least one air inlet sensor. The at least one air inlet sensor 41 may comprise a temperature sensor and/or a humidity sensor (for example an absolute humidity sensor and/or a relative humidity sensor).

As described above the apparatus 9 may receive a supplemental gas (for example oxygen via oxygen inlet port 28). The supplemental gas inlet may comprise at least one supplemental gas inlet sensor 42. The valve module 4001 as described above may be configured to operate to control the flow of supplemental gas (for example oxygen).

The at least one supplemental gas inlet sensor 42 may be part of the valve module 4001 or separate. In FIG. 7, the at least one supplemental gas inlet sensor 42 is shown as part of the valve module 4001 and located upstream of the valve 30. In some configurations, the sensor oxygen inlet sensor 42 may be provided separate from the valve module 4001, and upstream, or downstream of the valve module 4001. The at least one supplemental gas inlet sensor 42 may be configured to measure a property of the gases provided via the supplemental gas inlet.

The at least one supplemental gas inlet sensor 42 may comprise at least a pressure sensor. The pressure sensor may be configured to measure the pressure of the supplemental gas supply.

The apparatus 9 may also receive supplemental gas via an alternative supply inlet. The alternative supply inlet may be configured to receive supplemental gas at for example a lower pressure, or from an already controlled source (i.e. with flow control via an external valve).

The apparatus 9 may comprise one or more additional sensors 40. The additional sensors may be located within the apparatus (for example within the housing) and/or exposed to the ambient environment. The additional sensors may comprise a pressure sensor (for example an ambient pressure sensor). In some embodiments, the one or more additional sensors 40 are located at or near the valve module 4001.

As described above, the filter module 1001 receives the gases from the supplemental gas from the supplemental gas inlet and/or the alternative supply inlet, and the air from the air inlet.

As shown in FIG. 7, the flow generator 11 is pneumatically connected to the filter module. The flow generator 11 comprises a blower 11′ (as described in more detail elsewhere in the specification.) The flow generator 11 may comprise the motor and/or sensor module 400 as described in more detail above.

The flow generator 11 may comprise at least one blower sensor 43 configured to measure a characteristic of the blower. The at least one blower sensor 43 may be configured to measure a characteristic of the motor of the blower. The at least one blower sensor 43 may comprise a motor speed sensor.

The at least one blower sensor 43 may measure an electrical characteristic of the blower (for example the motor of the blower).

The at least one blower sensor 43 may be located as part of the blower, or located away from the blower on for example a control board (for example when the at least one blower sensor 43 measures an electrical characteristic of the blower).

The flow generator may also comprise at least one sensor 44 located downstream of the blower 11′ (as for example as shown in FIG. 7. In some configurations, the at least one sensor 44 is provided upstream of the blower. The at least one sensor 44 may be provided as part of the sensor module 400 (as described in more detail above). The at least one sensor 44 may comprise one or more of: at least one temperature sensor, at least one flow rate sensor (for example one or more ultrasonic transducers as described above), at least one pressure sensor (for example an absolute pressure sensor and/or a differential pressure sensor configured to measure the pressure in the gases flow path relative to ambient), at least one humidity sensor.

The at least one sensor 44 is located upstream of the humidifier (as shown in FIG. 7)

In some examples, the at least one sensor 44 comprises at least one flow sensor, at least one pressure sensor, at least one temperature sensor, and at least one humidity sensor.

The at least one pressure sensor may comprise a gauge pressure sensor (configured to measure a difference between the pressure of the flow of gases, and ambient pressure), and an absolute pressure sensor. In some examples as described above, the apparatus 9 may comprise an ambient pressure sensor

The at least one sensor 44 is configured to measure a property of the gases flow in the gases pathway downstream of the blower.

The sensor 3a as described above may be the blower sensor 43 and/or sensor 44.

IAs shown in FIG. 7, the apparatus 10 may also comprise at least one non return valve (NRV) 31 in the humidifier inlet. The non-return valve may be optional. The humidifier inlet may for example be fixed elbow 324 as described above.

The humidifier 12 as described in more detail above is pneumatically connected to the flow generator (for example via at least the fixed elbow 324). The humidifier heater may comprise at least one humidifier heater sensor 45. The at least one humidifier heater sensor 35 may be a temperature sensor and/or a humidifier heater power sensor configured to measure the power provided to the humidifier heater. The humidifier heater power sensor may be located away from the heater (for example on a control board).

As shown in FIG. 7, the apparatus 9 may also comprise at least one humidifier outlet sensor 46 located in the humidifier outlet. The humidifier outlet may be the elbow 325. The humidifier outlet sensor 46 may be one or more temperature sensors. The humidifier outlet sensor 46 is configured to measure a property of the gases flow in the humidifier outlet.

The sensor 3b as described above may be the humidifier heater sensor 45 and/or humidifier outlet sensor 46. Similarly, the sensor 3c may be the humidifier outlet sensor 46.

As described above, an inspiratory conduit 121 comprises a heater 120. The inspiratory conduit 121 may comprise at least one inspiratory conduit sensor 48. The at least one inspiratory conduit sensor 48 may be located at a patient end of inspiratory conduit 121 and configured to measure a property of the gases flow in the inspiratory conduit 121. The at least one inspiratory conduit sensor 48 may be a temperature sensor.

A power sensor 47 may also be provided to measure the power provided to the heater 120 of the inspiratory conduit 121. The power sensor 47 may be located away from the inspiratory conduit 121 (for example on a control board). The power sensor 47 may be part of the apparatus, and electrically connected to the heater 120 of the inspiratory conduit 121.

As described above, the respiratory system may be configured to deliver high flow therapy, Bubble CPAP therapy, variable flow CPAP, asynchronous Nasal Intermittent Positive Pressure Ventilation, synchronous Nasal Intermittent Positive Pressure Ventilation, NIV-NAVA therapy, HFOV therapy, volume-limited pressure control therapy, volume control ventilation therapy, and resuscitation therapy.

The breathing assistance apparatus may be changeable between a high flow therapy mode, a Bubble CPAP therapy mode, a variable flow CPAP mode, an asynchronous Nasal Intermittent Positive Pressure Ventilation mode, a synchronous Nasal Intermittent Positive Pressure Ventilation mode, a NIV-NAVA mode, a HFOV mode, a volume-limited pressure control mode, a volume control ventilation mode, and a resuscitation mode.

In the high flow therapy mode the breathing assistance apparatus is configured to provide high flow therapy

In the Bubble CPAP therapy mode the breathing assistance apparatus is configured to provide bubble CPAP therapy.

In the high flow therapy mode the breathing assistance apparatus is configured to provide high flow therapy,

In the Bubble CPAP therapy mode the breathing assistance apparatus is configured to provide bubble CPAP therapy,

In the variable flow CPAP mode the breathing assistance apparatus is configured to provide variable flow CPAP therapy,

In the asynchronous Nasal Intermittent Positive Pressure Ventilation mode the breathing assistance apparatus is configured to provide asynchronous Nasal Intermittent Positive Pressure Ventilation therapy, and

In the synchronous Nasal Intermittent Positive Pressure Ventilation mode the breathing assistance apparatus is configured to provide synchronous Nasal Intermittent Positive Pressure Ventilation therapy.

In the NIV-NAVA mode the breathing assistance apparatus is configured to provide NIV-NAVA therapy.

In the HFOV mode the breathing assistance apparatus is configured to provide HFOV therapy.

In the volume-limited pressure control mode the breathing assistance apparatus is configured to provide volume-limited pressure control therapy.

In the volume control ventilation mode, the breathing assistance apparatus is configured to provide volume control ventilation therapy.

In the resuscitation mode the breathing assistance apparatus is configured to provide resuscitation therapy.

The high flow therapy may be nasal high flow therapy.

In the high flow therapy mode the system comprises an unsealed patient interface coupled to the inspiratory conduit 121.

The unsealed patient interface may be a nasal cannula.

In use, the nasal cannula is positioned on the user's face to provide gases to the nares of the user.

The nasal cannula may comprise asymmetrical nasal delivery elements. For instance, the nasal cannula may comprise a first nasal delivery element and a second nasal delivery element. The first nasal delivery element may have a greater internal cross-sectional area than the second nasal delivery element. This difference in internal cross-sectional area may cause an asymmetrical flow or partial unidirectional flow of gases at the nares of a patient. This may be beneficial as it may for example, enhance dead-space clearance, reduce peak expiratory pressure, reduce noise, reduce resistance to flow, and thereby improve the quality of therapy. An interface with asymmetrical nasal delivery elements is described in PCT application PCT/NZ2014/000163, PCT publication WO2015020540, which is incorporated by reference in its entirety.

In the high flow mode the apparatus may be configured to deliver a set flow rate to the patient.

In the Bubble CPAP therapy mode the system comprises a sealed patient interface coupled to the inspiratory conduit 121, an expiratory conduit 130 coupled to the sealed patient interface. As described above, the sealed patient may be nasal mask, an oral mask, a full face mask, nasal pillows, or a cannula with sealing nasal prongs.

In the bubble CPAP mode the apparatus may be configured to deliver a set flow rate to the patient.

The expiratory conduit 130 is coupled to a pressure regulator to regulate pressure within the patient interface and/or the patient's airways.

As described in more detail above, the pressure regulator comprises a chamber with a column of water and the expiratory conduit 130 being submerged into the column of water. The pressure provided to the user being defined or being set by the depth the submersion of the expiratory conduit 130 within the column of water.

In the variable flow CPAP therapy mode the system comprises a sealed patient interface coupled to the inspiratory conduit 121, an expiratory conduit 130 coupled to the sealed patient interface.

In the variable flow CPAP mode the apparatus may be configured to deliver a set pressure to the patient.

For example the flow provided to the patient may vary to provide the set pressure.

The expiratory conduit 130 is coupled to a pressure regulator to regulate pressure within the patient interface and/or the patient's airways.

As described in more detail above, the pressure regulator comprises a expiratory orifice.

In the asynchronous and synchronous Nasal Intermittent Positive Pressure Ventilation mode therapy mode the system comprises a sealed patient interface coupled to the inspiratory conduit 121.

In some examples, in the NIV-NAVA mode, the HFOV mode, the volume-limited pressure control mode, the volume controlled ventilation mode, and/or the resuscitation mode, the system may comprise a sealed interface.

In some examples, an expiratory conduit 130 coupled to the sealed patient interface.

In some examples, the patient interface may comprise a vent.

As described above the apparatus may comprise an asynchronous Nasal Intermittent Positive Pressure Ventilation mode

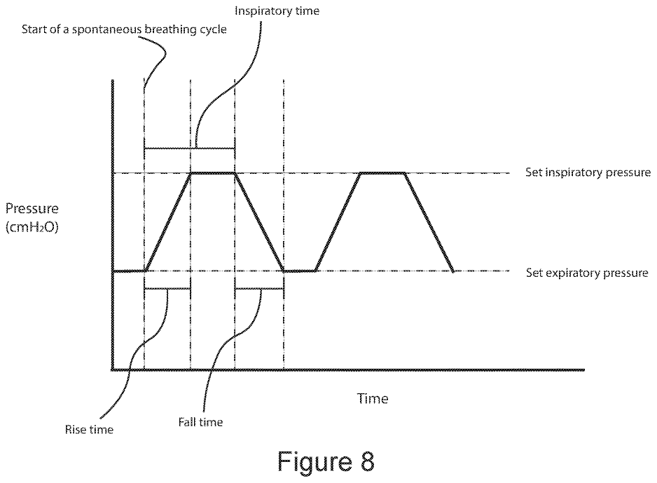

In the asynchronous Nasal Intermittent Positive Pressure Ventilation mode, the apparatus is configured to provide gases to the patient as a breathing cycle comprising a set inspiratory airway pressure to cause inspiration of the patient, and a set expiratory airway pressure to cause expiration of the patient. An example pressure provided to the patient is shown in FIG. 8.

The breathing cycle may have a set time period. The set breathing cycle time period may be set by the user (for example via the user interface 14).

In the asynchronous Nasal Intermittent Positive Pressure Ventilation mode, the apparatus 9 may be configured to provide gases to the patient at the set inspiratory airway pressure and the set expiratory airway pressure irrespective of detection of the start of a spontaneous breathing cycle for example as shown in FIG. 8. In other words, the provision of the set inspiratory airway pressure and the set expiratory airway pressure is independent of spontaneous breathing.

As described above the apparatus may comprise a synchronous Nasal Intermittent Positive Pressure Ventilation mode. In the synchronous Nasal Intermittent Positive Pressure Ventilation mode, the apparatus 9 may be configured to provide gases to the patient at a set inspiratory airway pressure on detection of the start a spontaneous breathing cycle. In other words, the provision of the set inspiratory airway pressure and the set expiratory airway pressure is based on spontaneous breathing of the patient. In some examples, of the start a spontaneous breathing cycle is the inhalation of a patient.

Breathing of the patient may serve as a trigger for the apparatus to provide the set inspiratory pressure. For example, detection of inspiration triggers the apparatus to provide the set inspiratory pressure. The apparatus may transition from the providing the set inspiratory pressure to providing the set expiratory pressure after a time period (as described in more detail below.)

In the synchronous Nasal Intermittent Positive Pressure Ventilation mode, the apparatus 9 may be configured to provide gases to the patient at a set expiratory airway pressure during expiration of the patient. In some examples, provision of the set expiratory airway pressure may be based on the start of expiration of the patient as detected by the apparatus 9, or as described in more detail below be based on a time period from detection of the start of a spontaneous breathing cycle.

In the synchronous Nasal Intermittent Positive Pressure Ventilation mode, the apparatus is configured to provide gases to the patient at a set inspiratory airway pressure during at least part of, or all of the inspiration of a patient. For example, in cases where the start of expiration is not detected (and the provision of the set expiratory airway pressure is based on a time after detection of the start of a spontaneous breathing cycle) the set inspiratory airway pressure may not be provided for the entire inspiration of the patient, or may be provided from longer than the inspiration of the patient.

In the synchronous Nasal Intermittent Positive Pressure Ventilation mode, the apparatus is configured to provide gases to the patient at a set expiratory airway pressure at: a predetermined time after detection of the start a spontaneous breathing cycle, or at a predetermined time after detection of an end of inhalation of a spontaneous breathing cycle.

In the synchronous Nasal Intermittent Positive Pressure Ventilation mode, the set inspiratory airway pressure is provided to the patient at a start of a spontaneous breath cycle, or after a predetermined time passes without detection of a start of a spontaneous breath cycle. This ensures that a patient receives a back-up breath if there is no spontaneous breathing detected.

The start of a spontaneous breath cycle may be based on a determined flow (for example measured by a flow rate sensor as described above) exceeding a spontaneous breath flow rate threshold.

In some examples, the spontaneous breath flow rate threshold is about 0.5 Litres/minute to about 7.0 Litres/minutes.

In some examples, the breathing cycle may be determined by a determined pressure (for example a measured pressure). For example, the start of a spontaneous breath cycle may be determined by a determined pressure decreasing below a pressure threshold.

As described above, the set inspiratory airway pressure may be provided after a predetermined time passes without detection of a start of a spontaneous breath cycle. The predetermined time without detection of a start of a spontaneous breath cycle may be based on a minimum breathing rate. The minimum breathing rate may be set by a user (for example via the user interface).

As shown in FIG. 8, in the asynchronous and synchronous Nasal Intermittent Positive Pressure Ventilation mode, the apparatus is configured to alternate in providing gases to the patient at the set inspiratory airway pressure during, and the set expiratory airway pressure.

The transition between the set inspiratory airway pressure during and the set expiratory airway may be for example linear.

As for example shown in FIG. 8, a transition from providing the set expiratory airway pressure to the set inspiratory airway pressure is defined by a rise time.

In some examples, the rise time is about 0.1 seconds to about 2 seconds.

As for example shown in FIG. 8, a transition from providing the set inspiratory airway pressure to the set expiratory airway pressure is defined by a fall time.

In some examples, the fall time is about 0.1 seconds to about 2 seconds.

As for example shown in FIG. 8, the set inspiratory airway pressure is provided after the rise time for a latter portion of an inspiration time. In some examples, the inspiration time is about 0.1 seconds to about 3 seconds.

As for example shown in FIG. 8, the inspiration time comprises the rise time and the time period for which the set inspiratory airway pressure is provided.

In some examples, the inspiration time may be set so as not to exceed half of a period of a breath cycle.

In the NIV-NAVA mode the system may be configured to control the flow and/or pressure of gas supplied to the patient based on the measurement of at least one physiological sensor attached to the patient.

In the NIV-NAVA mode the system may comprise a physiological sensor. Examples physiological sensors may include, but are not limited to, electrical diaphragm sensors or electromyography (EMG) sensors.

The physiological sensor may be in communication with the apparatus. The communication with the apparatus may be for instance a wired communication or a wireless communication (for example via a wireless interface).

The physiological sensor may measure a patient characteristic. For instance, the physiological sensor may measure patient breathing. In some configurations the physiological sensor may be configured to measure a parameter indicative of patient inhalation and/or exhalation. In some configurations the physiological sensor may be configured to detect the start of patient inhalation and/or patient exhalation. The physiological sensor may also measure other events in the breathing cycle. In some configurations the physiological sensor may be configured to output signals which are in anticipation of patient inhalation and/or patient exhalation i.e. before they happen.

In the NIV-NAVA mode the patient may be supplied with a flow of gas at a set inspiratory airway pressure during at least part of an inspiratory phase of a breathing cycle, and a set expiratory airway pressure during at least part of an expiratory phase of a breathing cycle. The delivery of the set inspiratory airway pressure and/or set expiratory airway pressure may be based on the measured patient characteristic, such as patient breathing. For instance, the delivery of the set inspiratory airway pressure may be synchronized with, or relative to at least the measurement of patient inhalation. Further, the delivery of the set expiratory airway pressure may be synchronized with, or relative to at least the measurement of patient exhalation. It will be appreciated that other measured parts of the breathing cycle may be used to control provision of the set inspiratory airway pressure and/or set expiratory airway pressure.

In the NIV-NAVA mode the apparatus may deliver the flow of gases invasively (for example via a tracheostomy or intubation) or non-invasively (i.e. via a nasal cannula or mask).

In the HFOV mode the system may be configured to deliver a set pressure level, with pressure oscillations superposed upon it.

The frequency of the superposed pressure oscillations may be very high (relative to the breathing cycle). For example, in some configurations there may be 300 to 900 cycles per minute. By way of further example, the system may be controlled to provide pressure oscillations at approximately 10 Hz for mature infants. By way of further example, the system may be controlled to provide pressure oscillations at about 3 Hz to about 6 Hz, or about 10 Hz to about 15 Hz, or about 12 Hz to about 15 Hz for example for preterm infants.

The pressure oscillations may be achieved by various techniques known by persons skilled in the art. For instance, in some configurations the pressure oscillations may be achieved by applying a vibrating speaker, a valving arrangement and/or a reciprocating diaphragm.

In some configurations, in a High Frequency Percussive Ventilation (HFPV) mode the set base pressure level may vary between a first base pressure level and a second base pressure level. The first base pressure level may be an inspiratory airway pressure and may be delivered during at least part of an inspiratory phase of a breathing cycle. The second base pressure level may be expiratory airway pressure and may be delivered during at least part of an expiratory phase of a breathing cycle. It will be appreciated that other measured parts of the breathing cycle may be used to control provision of the set inspiratory airway pressure and/or set expiratory airway pressure, such as to synchronize said provision with the start of inspiration and expiration, respectively.

In the NIV-NAVA mode the apparatus may be delivered invasively (for example via a tracheostomy or intubation) or non-invasively (i.e. via a nasal cannula or mask).

In the volume-limited pressure control mode the system may be configured to supply a patient with a flow of gas at a set pressure(s), but regulate the supply based on a delivered volume of gases to the patient.

The patient may be supplied with a flow of gas at a set inspiratory airway pressure during at least part of an inspiratory phase of a breathing cycle, and a set expiratory airway pressure during at least part of an expiratory phase of a breathing cycle (which may be for example a positive expiratory pressure). In some configurations the set inspiratory airway pressure may be the same as the set expiratory airway pressure. The set pressure may be a maximum set inspiratory airway pressure which may be set by a clinician.

The apparatus may be configured to monitor the delivered volume to the patient.

The apparatus may be configured to control the supply of the gas based on the delivered volume during inhalation. For instance, the apparatus may be configured to compare the monitored delivered volume to a set volume range or threshold. In some configurations, the apparatus may be configured to take a certain action if the monitored delivered volume is outside the set volume range and/or reaches/exceeds or is below the volume threshold. The volume threshold may for instance comprise a maximum volume threshold and/or a minimum volume threshold.

In some configurations if the delivered volume reaches or exceeds the maximum volume threshold, then the apparatus may take a certain action. The action may for instance be to allow exhalation and/or to cease providing pressure to the patient and/or decrease the set inspiratory airway pressure.

In some configurations if the delivered volume is below the minimum volume threshold, then the apparatus may take a certain action. The action may for instance be to increase the set inspiratory airway pressure which may increase the volume of gases delivered to the patient. The action may be taken on the same breath or a following breath.

The apparatus may also change the set inspiratory airway pressure based on the delivered volume during a breath. For example, if an estimated delivered volume is less than the minimum volume threshold then the apparatus may increase the set inspiratory airway pressure so as to target the delivered volume to the minimum volume threshold. Additionally, or alternatively, if an estimated delivered volume is greater than the maximum volume threshold then the apparatus may decrease the set inspiratory airway pressure so as to target the delivered volume to the maximum volume threshold.

In the volume controlled ventilation mode the apparatus may be configured to supply a patient with a flow of gas at a set volume per inhalation. For example, the apparatus may be configured to deliver the flow of gas at a set tidal volume and at a set flow rate.

In the volume controlled ventilation mode the apparatus may control a flow rate and/or a pressure, to meet the set volume during inhalation. For example, the apparatus may increase or decrease the flow rate and/or the pressure (for example set inspiratory pressure) to achieve the set volume during inhalation.

In some configurations the apparatus may comprise a set maximum flow rate that the apparatus may be unable to increase the flow of gases above.

In the volume-limited pressure control mode, and/or the volume controlled ventilation mode, the system may be configured to deliver invasive or non-invasive therapy. In non-invasive configurations the system may preferably comprise a proximal pressure and/or a flow sensor at the patient end of the system. This may improve therapy by enabling non-invasive volume-based control.

In non-invasive configurations of the synchronous Nasal Intermittent Positive Pressure Ventilation mode, the asynchronous Nasal Intermittent Positive Pressure Ventilation mode, the NIV-NAVA mode, the HFOV mode, the volume-limited pressure control mode and/or the volume controlled ventilation mode, the system may comprise an unsealed interface. In some configurations the unsealed interface may be configured to achieve an expiratory resistance to provide the required PEEP. For example, the unsealed interface may comprise for example a pressure relief valve or other pressure relief mechanism.

In non-invasive configurations of the synchronous Nasal Intermittent Positive Pressure Ventilation mode, the asynchronous Nasal Intermittent Positive Pressure Ventilation mode, the NIV-NAVA mode, the HFOV mode, the volume-limited pressure control mode and/or the volume controlled ventilation mode, the system may comprise a patient interface. The patient interface may for instance be any mask that a person skilled in the art would consider suitable for the delivery of non-invasive respiratory therapy. In some configurations, the patent interface may comprise a nasal chamber and an oral chamber. In some configurations, the nasal chamber and the oral chamber may be distinct. In some configurations, the interface may be a face mask, an oro-nasal mask, a nasal mask, a nasal pillow mask, or a nasal cannula.

In the resuscitation mode the apparatus may be configured to deliver a simulated breath to a patient for resuscitation purposes.

In some configurations the simulated breath may be implemented by adjusting the set pressure of the apparatus between a first and second pressure. The first pressure may be a set inspiratory pressure. The second pressure may be a set expiratory pressure. For instance, the first pressure could be a positive inspiratory pressure (PIP) and the second pressure could be a positive end expiratory pressure (PEEP). The pressure adjustment between the first and second pressures could therefore be configured to simulate a breath with the PIP being provided during an inhalation portion of the breath, and the PEEP being provided during an exhalation portion of the breath.

The pressure adjustment may be achieved by a trigger of the respiratory therapy system. The trigger may comprise for example a button, or a valve and/or aperture. A user or clinician may interact with the trigger to adjust the pressure between the first and the second pressures and thus simulate a breath.

In some configurations the trigger may be located on a component of the system, for example on a connector, or on the patient interface, or on the expiratory conduit.

In some configurations, the trigger may be a mechanical trigger for example a valve which has a positive end expiratory pressure (PEEP) outlet which a user may occlude to adjust the pressure to the second pressure, and unocclude (such that gases flow through the PEEP outlet) to adjust the pressure to the first pressure.

In some configurations, the trigger may be configured to transition the pressure between the first pressure and the second pressure in response to a signal being detected. The signal may be detected for example by a sensor. The sensor may be in communication with the apparatus.

The present system provides a single breathing assistance apparatus that can be used to deliver a number of therapy modes, while only the interface requiring changes (for example in transition to a high flow therapy). There are no changes in components on the gases supply side i.e. no changes in the gases supply components since a common respiratory apparatus can be used to deliver humidified gases.

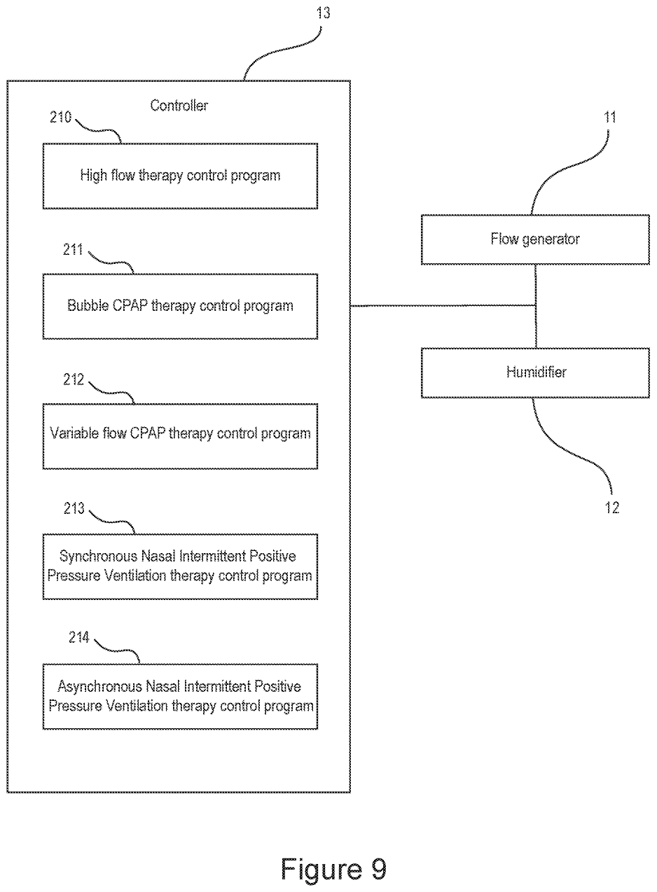

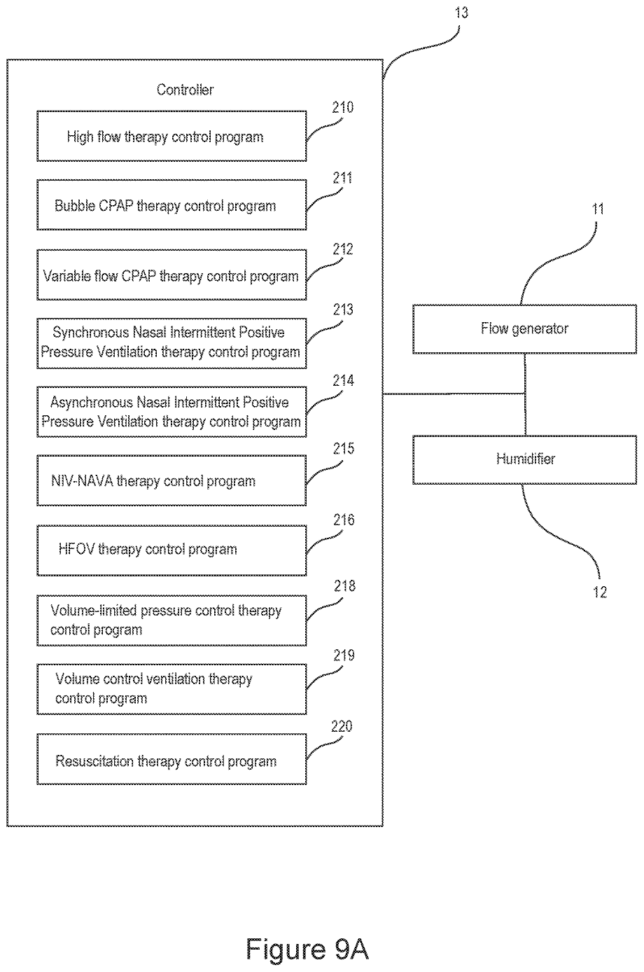

As shown in FIG. 9, the controller 13 may comprise a high flow therapy control program 210 associated with the high flow therapy mode.

As shown in FIG. 9, the controller 13 may comprise a bubble CPAP therapy control program 211 associated with the bubble CPAP therapy mode.

As shown in FIG. 9, the controller 13 may comprise a variable flow CPAP therapy control program 212 associated with the bubble CPAP therapy mode.

As shown in FIG. 9, the controller 13 may comprise a synchronous Nasal Intermittent Positive Pressure Ventilation mode therapy control program 213 associated with the bubble CPAP therapy mode.

As shown in FIG. 9, the controller 13 may comprise an asynchronous Nasal Intermittent Positive Pressure Ventilation mode therapy control program 214 associated with the bubble CPAP therapy mode.

As shown in FIG. 9A, the controller 13 may comprise a NIV-NAVA therapy control program 215 associated with the NIV-NAVA mode.

As shown in FIG. 9A, the controller 13 may comprise a HFOV therapy control program 216 associated with the HFOV mode.

As shown in FIG. 9A, the controller 13 may comprise a volume-limited pressure control therapy control program 218 associated with the volume-limited pressure control mode.

As shown in FIG. 9A, the controller 13 may comprise a volume control ventilation therapy control program 219 associated with the volume control ventilation mode.

As shown in FIG. 9A, the controller 13 may comprise a resuscitation therapy control program 220 associated with the resuscitation mode.

In some examples, each control program may have an associated separate controller, or be executed across a number of controllers.

The controller 13 is configured to select and apply the program 210, 211, 212, 213, 214 that corresponds to the selected mode of operation.

A user may be able to select a desired therapy mode from the plurality of therapy modes (for example via the user interface).

Each of the therapy control programs 210, 211, 212, 213, 214, 215, 216, 217, 218, 219, 220 defines corresponding therapy parameters.

The therapy parameters may comprise any one or combination of:
A set humidity level,
A set gases temperature (for example a temperature of the gases at a patient end of the conduit),
A set pressure (for example in variable CPAP mode) or set pressures (for example in the Nasal Intermittent Positive Pressure Ventilation modes),
A set flow rate (for example in high flow and/or bubble CPAP mode),

The therapy parameters may be based on treatment of neonatal and/or infant patients. For example, the set pressures and set flow rates may be suitable for use with neonatal and/or infant patients.

The humidity level may comprise one or more relative humidity, temperature or dew point set points to control the humidifier.

The set gases temperature for example a temperature of the gases at a patient end of the conduit, may be controlled by controlling the heater 120 of the inspiratory conduit 121 (as described in more detail above). The set gases temperature may be a fixed value, or be based on a temperature of the gases downstream of the flow generator (but upstream of the humidifier) and an offset. A gases property sensor being a temperature sensor being located at or near the patient end of the conduit may operate as a control input for the heater 120 of the inspiratory conduit 121, to control the temperature of the gases to the set gases temperature.

The therapy parameters may also comprise any of the parameters discussed above with respect to each therapy.

The therapy control programs may also define alarms:
which alarms apply to the therapy mode associated with the control program,
alarm conditions associated the one or more alarms
any associated alarm thresholds associated the one or more alarms.

The controller 13 may be configured to generate one or more alarms.

The alarms may be generated based on an output at least one gases property sensor. For example the alarms may be generated based on a determined flow rate and/or determined pressure of the gases in the gases flow path.

The alarms may comprise any one or combination of:
An over pressure alarm,
An elevated pressure alarm,
A low pressure alarm,
An excessive leak alarm,
A blockage alarm,
An apnea alarm.
An intermittent or no bubbling alarm,
A condensation alarm,
An incorrect hardware alarm,
A prong dislodge alarm,
A loss of physiological signal alarm,
A high/low breath rate alarm,
A tidal volume alarm,
An SpO2 alarm.
An exhaled CO2 alarm.

It will be appreciated that each of the alarms may be associated with one or more of the plurality of therapy modes. The alarms may have different alarm conditions (for example thresholds) for each therapy mode.

In response to one or more of the alarms being generated the controller 13 may be configured to undertake one or a combination of the following:
Disable the flow generator, or reduce the output of the flow generator,
Disable a heater of the humidifier, or reduce the output of the heater of the humidifier,
Disable a heater of an attached conduit (for example the inspiratory conduit and/or the expiratory conduit, or reduce the output of the heater of the conduit(s).
Display a message on a display,
Generate an audio warning
Generate a visual warning.

Examples of alarms for some specific modes are described below, however it will be appreciated that these are just examples and are non-exhaustive.

In the variable flow CPAP therapy mode the one or more alarms comprise:
a) An over pressure alarm,
b) An elevated pressure alarm,
c) A low pressure alarm,
d) An excessive leak alarm,
e) A blockage alarm,
f) An apnea alarm,
g) Any combination of a)-f).

In the synchronous Nasal Intermittent Positive Pressure Ventilation therapy mode and/or the asynchronous Nasal Intermittent Positive Pressure Ventilation therapy mode the one or more alarms comprise:
a) An over pressure alarm,
b) An elevated pressure alarm,
c) A low pressure alarm,
d) An excessive leak alarm,
e) A blockage alarm,
f) An apnea alarm,
g) Any combination of a)-f).

In the bubble CPAP mode the one or more alarms may comprise an intermittent or no bubbling alarm.

In the NIV-NAVA mode the one or more alarms may comprise a loss of physiological signal alarm.

In the volume-limited pressure control mode the one or more alarms comprise a tidal volume alarm.

In the high flow mode, the one or more alarms may comprise a prong dislodge alarm.

The over pressure alarm may be generated when a determined pressure is above a threshold. The over pressure alarm may protect a patient from being delivered over pressure and aid in preventing potential patient harm for example from barotrauma.

The over pressure alarm may be a high severity and so in response to the over pressure alarm being activated the apparatus may be shut down to prevent harm to the patient. In some examples, the apparatus 9 may need to be power cycled to clear the alarm.

The determined pressure may be the pressure at a location in the gases flow path for example at or near a patient, or the end of an inspiratory conduit.

As described above, the over pressure alarm may be active in the variable flow CPAP therapy mode and/or the synchronous Nasal Intermittent Positive Pressure Ventilation therapy mode and/or. the asynchronous Nasal Intermittent Positive Pressure Ventilation therapy.

The over pressure alarm may be generated when the determined pressure is greater than an over pressure threshold.

The over pressure alarm may be generated when the determined pressure is greater than an over pressure threshold for an over pressure time period. In some examples, the over pressure time period is about 1 second.

The over pressure threshold for the over pressure alarm associated with the variable flow CPAP mode may be about 50 cmH2O to about 60 cmH2O, or about 55 cmH2O.

The over pressure threshold may be settable by a user (for example via a user interface).

In some configurations the over pressure threshold may be adjustable incrementally. For example, the over pressure threshold may be adjustable by increments of ½ cmH2O.

In some configurations the over pressure threshold may be adjustable within an allowable range.

With reference to the over pressure alarm, when the over pressure alarm is generated, the controller may be configured to:
a) Disable the flow generator,
b) Disable a heater of the humidifier,
c) Disable a heater of an attached conduit,
d) Display a message on a display,
e) Any combination of a)-d).

The elevated pressure alarm may be generated when a determined pressure is above a threshold.

The elevated pressure alarm may provide a warning to a clinician that the pressure being provided is too high, and the patient and circuit should be checked.

The determined pressure may be the pressure at a location in the gases flow path for example at or near a patient, or the end of an inspiratory conduit.

In some examples, the elevated pressure alarm may be indicative of a blockage or partial blockage in the gases flow path.

The elevated pressure alarm may be of particular use in a neonatal application, as elevated pressures may cause damage in neonates more quickly than in other patient types. The elevated pressure alarm may provide a warning before the pressure gets to unsafe levels—so that a clinician can address any issues.

As described above, the elevated pressure alarm may be active in the variable flow CPAP mode, the synchronous Nasal Intermittent Positive Pressure Ventilation therapy mode and the asynchronous Nasal Intermittent Positive Pressure Ventilation therapy.

The elevated pressure alarm may be generated when the determined pressure is greater than an elevated pressure threshold associated with one or more of the plurality of therapy modes.

The elevated pressure alarm may be generated when the determined pressure is greater than an elevated pressure threshold for an elevated pressure time period.

In some examples, the elevated pressure time period is greater than an over pressure time period. In some examples, the elevated pressure time period is about 5 seconds.

The elevated pressure threshold may be lower than a or the over pressure threshold. The elevated pressure may therefore be a lower severity alarm.

In the variable flow CPAP mode, the elevated pressure threshold is based on a or the set pressure of the variable flow CPAP mode. For example the elevated pressure threshold is relative to the set pressure delivered to the patient.

In some examples, in the variable flow CPAP mode, the elevated pressure threshold is greater than a or the set pressure of the variable flow CPAP mode by about 3 cmH2O to about 7 cmH2O, or about 5 cmH2O.

As described above, different therapy modes may have different alarm thresholds.

In the synchronous Nasal Intermittent Positive Pressure Ventilation mode and/or the asynchronous Nasal Intermittent Positive Pressure Ventilation mode, the elevated pressure threshold is greater than the set inspiratory airway pressure.

In some examples, in the synchronous Nasal Intermittent Positive Pressure Ventilation mode and/or the asynchronous Nasal Intermittent Positive Pressure Ventilation mode, the elevated pressure threshold is greater than a or the set inspiratory airway pressure by about 3 cmH2O to about 7 cmH2O, or about 5 cmH2O.

In some configurations the elevated pressure threshold may be adjustable incrementally. For example, the elevated pressure threshold may be adjustable by increments of ½ cmH2O.

In some configurations the elevated pressure threshold may be adjustable within an allowable range.

With reference to the elevated pressure alarm, when the elevated pressure alarm is generated, the controller is configured to:
a) Disable the flow generator,
b) Disable a heater of the humidifier,
c) Disable a heater of an attached conduit,
d) Display a message on a display,
e) Generate an audio warning
f) Generate a visual warning
g) Any combination of a)-f).

The low pressure alarm may be generated when a determined pressure is below a threshold.

The low pressure alarm may provide a warning to a clinician that the pressure being provided is too low, and the patient and circuit should be checked (for example a component of the circuit may be improperly connected, and/or misconnected). The low pressure alarm may provide warning to a clinician that therapy is not being provided.

The determined pressure may be the pressure at a location in the gases flow path for example at or near a patient, or the end of an inspiratory conduit.

As described above, the low pressure alarm may be active in the variable flow CPAP mode, the synchronous Nasal Intermittent Positive Pressure Ventilation therapy mode and/or the asynchronous Nasal

The low pressure alarm may be generated when the determined pressure is less than a low pressure threshold associated with one or more of the plurality of therapy modes.

The low pressure alarm may be generated when the determined pressure is lower than an low pressure threshold for an low pressure time period. In some examples, the elevated pressure time period is about 10 seconds.

In the variable flow CPAP mode, the low pressure threshold is based on a or the set pressure of the variable flow CPAP mode.

In some examples, in the variable flow CPAP mode, the elevated pressure threshold is less than a or the set pressure of the variable flow CPAP mode by about 1 cmH2O to about 3 cmH2O, or about 1 cmH2O.

In the synchronous Nasal Intermittent Positive Pressure Ventilation mode and/or the asynchronous Nasal Intermittent Positive Pressure Ventilation mode, the low pressure threshold may be less than the set expiratory airway pressure.

In some examples, in the synchronous Nasal Intermittent Positive Pressure Ventilation mode and/or the asynchronous Nasal Intermittent Positive Pressure Ventilation mode, the low pressure threshold is less than a or the set expiratory airway pressure by about 1 cmH2O to about 3 cmH2O, or about 1 cmH2O.

In some configurations the low pressure threshold may be adjustable incrementally. For example, the low pressure threshold may be adjustable by increments of ½ cmH2O.

In some configurations the low pressure threshold may be adjustable within an allowable range.

With reference to the low pressure alarm, when the low pressure alarm is generated, the controller is configured to:
a) Disable a heater of the humidifier,
b) Disable a heater of an attached conduit,
c) Display a message on a display,
d) Any combination of a)-c).

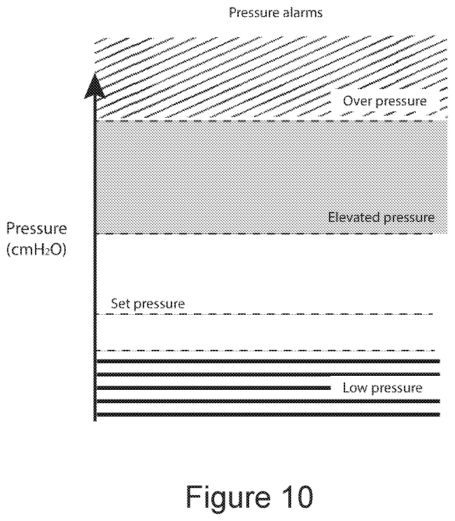

FIG. 10 sets out an example of the various pressure alarms and their relationships to each other. It will be appreciated that the thresholds for each therapy mode may be different.

The excessive leak alarm may be generated when a determined flow rate is above a threshold.

The excessive leak alarm may provide a warning to a clinician that the flow rate being provided is too high for the conditions of the system, and the patient and circuit should be checked for leaks (for example loose connections, or an incorrectly sized or fitted interface.

The determined flow rate may be a flow rate at the flow generator, or the flow rate at a location in the gases flow path for example at or near a patient, or the end of an inspiratory conduit.

The excessive leak alarm may be generated when the determined flow is greater than a excessive leak threshold associated with one or more of the plurality of therapy modes.

Wherein the excessive leak alarm associated is generated when the determined flow rate is greater than an excessive leak threshold for an excessive leak time period. In some examples, the excessive time period is about 10 seconds.

The excessive leak threshold may be based on a or the determined pressure

The determined pressure in the gases flow path is a pressure at or near the patient and/or at the end of an inspiratory conduit.

The excessive leak threshold may be based on a user settable variable indicative of an acceptable unintentional leak flow rate.

The excessive leak threshold may be for example a flow rate above which the leak must be too large for given conditions of the system (i.e. determined pressure).

In some configurations the system may be utilised with at least one mask. The system may further comprise a pressure-flow curve specific associated with each of the at least one masks. Each specific pressure-flow curve may enable detection of unintentional leaks for the given at least one mask. The pressure-flow curve may be used so that if the combination of pressure and flow measured from one or more sensors is above the curve this may indicate that there is leak (for example a leak above an excessive leak threshold).

The pressure-flow curves may be for instance preprogrammed into the system.

The user may for example enter an input indicative of the allowed leak at a particular pressure (for example 5 litres/minutes @ 5 cmH2O.) The controller may then use this to calculate an allowable leak conductance (i.e. allowed leak divided by the square root of the particular pressure. The particular pressure may be predefined or user settable.

The excessive leak threshold may be based on a flow through the pressure regulator and a flow caused by the leak.

Wherein the flow through the pressure regulator is based on a conductance of the pressure regulator and a determined pressure. The conductance may define a relationship between the flow rate and square root of the determined pressure of the pressure regulator.

The flow caused by leak is a function of the allowed leak and the set pressure.

$$( \frac{allowed leak}{\sqrt{particular pressure}} + \begin{matrix}Excessive leak threshold ∝ \\ {Conductance}_{Pressure regulator}\end{matrix} ) \sqrt{determined pressure}$$

The excessive leak alarm may be cleared when the determined flow rate is lower than an excessive leak threshold.

The excessive leak alarm may be cleared when the determined flow rate is lower than an excessive leak threshold, for an excessive leak time clear period. In some examples, the excessive leak time clear period about 3 seconds.

In some examples once the alarm has been generated, the alarm cannot be cleared for a predetermined time, wherein the predetermined time is about 5 seconds.

With reference to the excessive leak alarm, when the elevated pressure alarm is generated, the controller is configured to:
a) Display a message on a display,
b) Generate an audio warning
c) Generate a visual warning
d) Any combination of a)-c).

In some examples, in response to the excessive leak alarm being generated, the apparatus may transition to a flow control mode (as described in more detail below).

The prong dislodge alarm may be generated when a prong dislodgement leak is detected.

The apparatus may be configured to recognize a flow rate over a prong dislodgement threshold for a given pressure as being indicative of a prong dislodgement.

The flow rate of the prong dislodgement threshold may be different depending on the pressure of the delivered gas.

In some configurations the prong dislodge alarm is only generated when the measured flow rate increases over the prong dislodgement threshold for a predetermined period.

In some configurations. the controller may store a pressure-flow curve which defines a prong dislodgement threshold being indicative of a prong dislodgement leak. If the measured pressure and flow is above this curve then this may be indicative of a prong dislodgement leak.

The prong dislodgement threshold may be set by a user.

In some examples, in response to the prong dislodge alarm being generated, the apparatus may transition to a flow control mode.

The prong dislodge alarm may be stopped manually by a user and/or may be stopped automatically and/or semi-automatically. For example, the prong dislodge alarm may be stopped if no prong dislodgment is detected for a certain period.

The blockage alarm may be generated when a determined flow rate is below a threshold.

The blockage alarm may provide a warning to a clinician that the flow rate being provided is too low, and the patient and circuit should be checked for blockages (for example any occlusions in the conduits and/or kinked conduits).

The determined flow rate may be a flow rate at the flow generator, or the flow rate at a location in the gases flow path for example at or near a patient, or the end of an inspiratory conduit.

The blockage alarm may be generated when the determined flow is less than an blockage threshold associated with one or more of the plurality of therapy modes.

The blockage alarm may be generated when the determined flow rate is less than a blockage threshold for a blockage time period. In some examples, the blockage time period is about 10 seconds.

The blockage threshold may be based on a or the determined pressure.

The determined pressure in the gases flow path may be a pressure at or near the patient and/or at a patient end of an inspiratory conduit.

The blockage alarm may be cleared when the determined flow rate is lower than a blockage threshold.

The blockage alarm may be cleared when the determined flow rate is lower than a blockage threshold for a blockage time clear period. In some examples, the blockage time clear period about 10 seconds.

With reference to the blockage alarm when the blockage alarm is generated, the controller is configured to:
a) Disable a heater of the humidifier,
b) Disable a heater of an attached conduit,
c) Display a message on a display,
d) Any combination of a)-c).

The apnea alarm may be generated when a patient is not breathing.

The apnea alarm may provide a warning to a clinician that the patient is not breathing (for example is apneic), and the patient and circuit should be checked.

The apnea alarm is triggered when there are no breathing cycles detected.

In the asynchronous Nasal Intermittent Positive Pressure Ventilation mode, the apnea alarm may be triggered when no breathing cycles are provided to the patient.

The apnea alarm may be triggered when there are no breathing cycles detected, within a apnea time period. In some examples, the apnea time period is about 5 seconds to about 60 seconds.

The breathing cycle may be detected based on the detection of the start of a spontaneous breathing cycle and/or in asynchronous Nasal Intermittent Positive Pressure Ventilation mode when a breathing cycle is provided to the patient to cause inspiration and expiration.

The breathing cycles may be detected based on a determined flow and/or a determined pressure.

The breathing cycles are detected based on detection of a positive peak followed by a negative peak of the determined flow and/or a determined pressure.

The breathing cycles may be detected based on detection of a positive peak followed by a negative peak, wherein the difference between the amplitude of the positive peak and the negative peak is greater than a breath amplitude threshold.

In some examples, the breath amplitude threshold is about 5 to about 200 mL.

The apnea alarm is cleared when a breathing cycle is detected.

In some examples, once the apnea alarm has been generated, the alarm cannot be cleared for a predetermined time, wherein the predetermined time is about 5 seconds.

With reference to the apnea alarm, when the apnea alarm is generated, the controller is configured to:
a) Display a message on a display,
b) Generate an audio warning
c) Generate a visual warning
d) Any combination of a)-c).

The intermittent or no bubbling alarm may be generated when bubbling is intermittent and/or has ceased.

Bubbling may be measured by one or more sensors in the system. For instance, when the system is configured to be in the bubble CPAP mode. The sensor may be a flow and/or pressure sensor located in the apparatus, and/or in or proximal to the water column. As discussed in more detail above, bubbling in the water column may be generated by expired gases emitted from an expiratory tube that may be submerged in the water column.

Detection of bubbling may be based on a measured flow and/or pressure. Detection of bubbling is disclosed in PCT application no. PCT/IB2021/058563, PCT publication number WO2022/058982 which is incorporated by reference in its entirety.

The intermittent or no bubbling alarm may provide a warning to a clinician that there is intermittent bubbling and/or that there is no bubbling. This may indicate that inspiratory demand is not being met.

The intermittent or no bubbling alarm may be generated when a variable indicative of bubbling falls below a threshold.

In some configurations the intermittent or no bubbling alarm may be generated when the variable indicative of bubbling falls below a bubbling threshold for a predetermined period.

The bubbling threshold and/or the predetermined period may be set by the user or may be generated automatically and/or semi-automatically.

The intermittent or no bubbling alarm may be stopped manually by a user and/or may be stopped automatically and/or semi-automatically. For example, the alarm may be stopped if the variable indicative of bubbling is above the bubbling threshold for a certain period.

The condensation alarm may be generated when condensation is detected in the system.

The condensation alarm may provide a warning to a clinician that there is condensation in the system.

The breathing assistance system and/or apparatus may comprise a condensation detector configured to measure condensation. The condensation detector may be positioned in an appropriate location in the system and/or apparatus. In some configurations the condensation detector may be positioned downstream of the humidifier. For example, the condensation detector may be positioned in the breathing tube.

The condensation alarm may be generated when the condensation detector detects condensation. In some configurations, the condensation alarm may be generated when the measured condensation increases above a condensation threshold.

The condensation alarm may be generated based on a sensor signal indicative of the amount of moisture present in a component of the system (for example a conduit or other component of the system). The sensor may for example be a first electrically conductive element and a second electrically conductive element, and the sensor signal may be generated using one or more of the at least first and second electrically conductive elements. The first electrically conductive element and a second electrically conductive element may extend down a length of the component when for example the component is a tube. Further details of the condensation alarm, and how the alarm might be generated are disclosed in PCT application no. PCT/IB2021/020027, PCT publication number WO2021229307, and PCT application no. PCT/IB2022/060952, PCT publication number WO2023084492 which are incorporated by reference in its entirety.

In some configurations the condensation alarm may be generated when the measured condensation is above a condensation threshold for a predetermined period.

The condensation threshold and/or the predetermined period may be set by the user, or may be generated automatically, and/or semi-automatically.

The condensation alarm may be stopped manually by a user and/or may be stopped automatically and/or semi-automatically. For example, the condensation alarm may be stopped if the measured condensation is below the condensation threshold for a certain period.

The incorrect hardware alarm may be generated when connection of incorrect hardware is detected. For example, the incorrect hardware alarm may be generated if an incompatible interface or tube is connected to the system.

The incorrect hardware alarm may provide a warning to a clinician that incorrect hardware has been connected to the system. Using incorrect hardware is problematic as it may prevent the system from delivering safe and effective therapy.

What hardware is considered the correct hardware may depend on the present mode of the breathing assistance apparatus. For example, a certain interface may be the correct hardware for one mode and the incorrect hardware for another mode.

In some configurations the incorrect hardware alarm may be generated based on a comparison of the measured operational parameters of the connected hardware, with the known operational parameters of the correct hardware. If for example the measured operational parameters do not correspond with the known operational parameters, then the incorrect hardware alarm may be generated.

In some configurations the operational parameters may comprise flow and/or pressure characteristics. For example. the apparatus may measure the flow or pressure characteristics of the connected hardware. The apparatus may compare the measured flow and/or pressure characteristics to the known flow and/or pressure characteristics of the correct hardware. If the measured and known characteristics do not correspond then the incorrect hardware alarm may be generated.

In some configurations the correct hardware operational parameters for each mode may be pre-programmed into the system. For instance, the correct hardware operational parameters may be entered manually. In some configurations the correct hardware operational parameters could be entered into the system automatically (for example from a database located on a server).

The system may be configured to be non-operational (for example not providing gases to the patient, and turning off one or more parts of the apparatus e.g. the heater of the humidifier) whilst the incorrect hardware alarm has been activated. This may be beneficial as it prevents patients from receiving unsafe or ineffective therapy.

In some configurations the alarm may be stopped by the removal of the incorrect hardware.

The loss of physiological signal alarm may be generated when the connection between a physiological sensor and the apparatus has been lost.

As discussed above, in some configurations the patient may be equipped with at least one physiological sensor. If the apparatus ceases to receive a signal from the at least one physiological sensor, the loss of physiological signal alarm may be generated alerting a clinician of a loss of connection with the at least one physiological sensor. For example, in the NIV-NAVA mode at least one physiological sensor may be an electrical diaphragm sensor, and/or an electromyogram sensor attached to the patient. These sensors may have a wired or wireless connection with the apparatus. If the apparatus ceases to recognize a wired or wireless connection from the sensors then the loss of physiological signal alarm may be generated.

The loss of physiological signal alarm may be stopped manually, and/or semi-automatically, and/or automatically. For example, if the signal with the at least one physiological sensors is regained the alarm may be stopped.

The system may comprise various parameter sensors optionally additionally to the sensors described above, which are configured to directly or indirectly measure a signal indicative of a patient parameter. For example, the various parameter sensors may comprise a breath rate sensor, a tidal volume sensor, a SpO2 sensor, and/or an exhaled CO2 sensor.

The breath rate sensor may be configured to measure breath rate. The breath rate sensor may be provided at various locations within the system. For instance, the breath rate sensor may be provided on the apparatus, and/or the breathing tube, and/or the patient interface. Additionally, or alternatively the breath rate sensor may be provided on the patient, for instance the breath rate sensor may measure expansion and contraction of the patient's diaphragm. In some configurations, breath rate may be determined from flow and/or pressure measurements (for example from the flow rate sensor and/or the pressure sensor as described in more detail elsewhere in the specification).

The tidal volume sensor measures tidal volume. In some configurations the tidal volume sensor measures tidal volume by comparing the flow in the inspiratory limb to the flow in the expiratory limb. In some configurations, the tidal volume may be determined by integration of a flow rate signal over time (for example the flow rate signal being from a flow rate sensor as described elsewhere in the specification).

The SpO2 sensor (for example as a patient sensor) measures the SpO2 of the patient. In some configurations the SpO2 sensor may be disposed on a patient. For instance, the SpO2 sensor could be a pulse oximeter, and the pulse oximeter could be positioned to be in contact with the patient's skin, for example, the patient's fingertip and/or on the patient's face.

The exhaled CO2 sensor measures the amount of CO2 exhaled by the patient. In some configurations the exhaled CO2 sensor may be located near a patient's airway. In some configurations the exhaled CO2 sensor may be located at the expiratory tube of the apparatus.

In some configurations the apparatus may be configured to make adjustments to therapy parameters based on the measurements of the various parameter sensors.

The high/low breath rate alarm may be generated when the measured breath rate is considered to be too high or too low.

The high/low breath rate alarm may provide a warning to a clinician that the patient is breathing at a rate considered too high or too low.

The high/low breath rate alarm may be generated when the system determines the breath rate above a high breath rate threshold or below a low breath rate threshold. In some configurations the high/low breath rate alarm may be generated when the breath rate is above/below the high/low breath thresholds for a predetermined period.

In some configurations the high/low breath rate alarm may be generated when the breath rate increases greater than a breath rate increase threshold and/or the breath rate decreases greater than a breath rate decrease threshold. This may be indicative of patient deterioration.

The high and low breath rate thresholds and/or the predetermined period may be set by the user or may be generated automatically and/or semi-automatically.

The high/low breath rate alarm may be stopped manually by a user and/or may be stopped automatically and/or semi-automatically. For example, the alarm may be stopped if the measured breath rate is in a safe range for a certain period.

The tidal volume alarm may be generated when tidal volume increases or decreases out of a safe range. For instance, the safe range may be defined by a lower or upper tidal volume threshold. If the measured tidal volume increases or decreases past the thresholds, outside of the safe range, the tidal volume alarm may be generated to warn the clinician of unsafe tidal volume or insufficient pressure.

The measured tidal volume may be based on measurements made by the tidal volume sensors.

The tidal volume alarm may be stopped manually by a user and/or may be stopped automatically and/or semi-automatically. For example, the tidal volume alarm may be stopped if the measured tidal volume is in the safe range for a certain period.

The SpO2 alarm may be generated when the measured SpO2 is considered too high or too low.

The SpO2 alarm may provide a warning to a clinician that the patient has SpO2 levels which are considered too high or too low.

The SpO2 alarm may be generated when the SpO2 sensor detects SpO2 levels above a high SpO2 threshold or below a low SpO2 threshold. In some configurations the SpO2 alarm may be generated when the measured SpO2 is above/below the SpO2 thresholds for a predetermined period.

The high and low SpO2 thresholds and/or the predetermined period may be set by the user or may be generated automatically and/or semi-automatically.

The SpO2 alarm may be stopped manually by a user and/or may be stopped automatically and/or semi-automatically. For example, the alarm may be stopped if the measured SpO2 is in a safe range for a certain period.

The exhaled CO2 alarm may be generated when the measured exhaled CO2 is considered too high.

The exhaled CO2 alarm may provide a warning to a clinician that the patient has CO2 levels considered too high.

The exhaled CO2 alarm may be generated when the exhaled CO2 sensor detects exhaled CO2 levels above an exhaled CO2 threshold. In some configurations the exhaled CO2 alarm may be generated when the measured exhaled CO2 is above the exhaled CO2 threshold for a predetermined period.

The exhaled CO2 threshold and/or the predetermined period may be set by the user or may be generated automatically and/or semi-automatically.

The exhaled CO2 alarm may be stopped manually by a user and/or may be stopped automatically and/or semi-automatically. For example, the alarm may be stopped if the exhaled CO2 is beneath the exhaled CO2 threshold for a certain period.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication. For example, actions such as “controlling a motor speed” include “instructing controlling of a motor speed.”

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. In some embodiments, the computer system may be a cloud-based computing system whose processing resources are shared by multiple distinct business entities or other users.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A breathing assistance apparatus configured to deliver respiratory therapy to a patient, the breathing assistance apparatus comprising:
a flow generator, and a humidifier in fluid communication with the flow generator,
a controller configured to control the breathing assistance apparatus,
wherein the controller is configured to be in communication with at least one gases property sensor, wherein the controller is configured to calculate a determined flow rate and/or a determined pressure of the gases in a gases flow pathway based on an least an output of the at least one gases property sensor,
wherein the controller is configured to generate one or more alarms based on the determined flow rate and/or determined pressure, wherein the one or more alarms comprise one or more of:
an over pressure alarm,
an elevated pressure alarm,
a low pressure alarm,
an excessive leak alarm,
a blockage alarm, or
an apnea alarm.

2. The breathing assistance apparatus of claim 1 wherein the one or more alarms further comprise one or more of:
an intermittent or no bubbling alarm,
a condensation alarm,
an incorrect hardware alarm,
a prong dislodge alarm,
a loss of physiological signal alarm,
a high/low breath rate alarm,
a tidal volume alarm,
an SpO2 alarm, or
an exhaled CO2 alarm.

3. (canceled)

4. (canceled)

5. (canceled)

6. The breathing assistance apparatus of claim 1, wherein when the one or more alarms is generated, the controller is configured to do one or more of:
disable the flow generator,
disable a heater of the humidifier,
disable a heater of an attached conduit,
display a message on a display,
generate an audio warning, or
generate a visual warning.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The breathing assistance apparatus of claim 1, wherein the over pressure alarm is generated when a or the determined pressure is greater than an over pressure threshold.

28. The breathing assistance apparatus of claim 1, wherein an over pressure alarm is generated when a determined pressure is greater than an over pressure threshold for an over pressure time period.

29. (canceled)

30. (canceled)

31. (canceled)

32. (canceled)

33. The breathing assistance apparatus of claim 1, wherein an elevated pressure alarm is generated when the determined pressure is greater than an elevated pressure threshold for an elevated pressure time period.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The breathing assistance apparatus of claim 1, wherein the excessive leak alarm is generated when the determined flow rate is greater than an excessive leak threshold for an excessive leak time period.

46. The breathing assistance apparatus of claim 45, wherein the excessive leak threshold is based on a or the determined pressure.

47. The breathing assistance apparatus of claim 46, wherein the determined pressure in the gases flow pathway is a pressure at or near the patient and/or at an end of an inspiratory conduit.

48. The breathing assistance apparatus of any one of claim 45, wherein the excessive leak threshold is based on a user settable variable indicative of an acceptable unintentional leak flow rate.

49. The breathing assistance apparatus of claim 48, wherein the acceptable unintentional leak flow rate is based on an allowed leak flow rate at a particular pressure.

50. The breathing assistance apparatus of claim 49, wherein the excessive leak threshold is based on a flow through a pressure regulator and a flow caused by leak.

51. The breathing assistance apparatus of claim 50, wherein the flow through the pressure regulator is based on a conductance of the pressure regulator and a set pressure.

52. The breathing assistance apparatus of claim 50, wherein the flow caused by leak is a function of the allowed leak set by the user and the particular pressure.

53. The breathing assistance apparatus of claim 45, wherein the excessive leak alarm is cleared when a or the determined flow rate is lower than the excessive leak threshold.

54. The breathing assistance apparatus of claim 53, wherein the excessive leak alarm is cleared when the determined flow rate is lower than the excessive leak threshold, for an excessive leak time clear period.

55. (canceled)

56. The breathing assistance apparatus of claim 1, wherein a or the blockage alarm is generated when the determined flow rate is less than a blockage threshold for a blockage time period.

57. The breathing assistance apparatus of claim 56, wherein the blockage threshold is based on a or the determined pressure.

58. The breathing assistance apparatus of claim 56, wherein the blockage alarm is cleared when the determined flow rate is lower than the blockage threshold.

59. (canceled)

60. The breathing assistance apparatus of claim 1, wherein a or the apnea alarm is triggered when there are no breathing cycles detected.

61. (canceled)

62. (canceled)

63. (canceled)

64. (canceled)

65. (canceled)
